(19) United States
(12) Patent Application Publication
(10) Pub. No.: US 2025/0281509 A1
(43) Pub. Date: Sep. 11, 2025

(54) SEQUENTIAL HORMONE THERAPY TO IMPROVE SURVIVAL AND ENHANCE RESPONSE TO IMMUNE THERAPY IN MEN WITH PROSTATE CANCER

(71) Applicant: The Johns Hopkins University, Baltimore, MD (US)

(72) Inventors: Samuel R. Denmeade, Ellicott City, MD (US); John T. Isaacs, Phoenix, MD (US); Emmanuel S. Antonarakis, Silver Spring, MD (US); Sushant Kachhap, Baltimore, MD (US); Mark Christopher Markowski, Baltimore, MD (US)

(21) Appl. No.: 18/556,964

(22) PCT Filed: Apr. 26, 2022
(86) PCT No.: PCT/US2022/026376

§ 371 Date: Oct. 24, 2023

Related U.S. Application Data
(60) Provisional application No. 63/179,709, filed on Apr. 26, 2021.

(51) Int. Cl. A61K 31/58; A61K 31/4152; A61K 31/568; A61K 38/09; A61K 45/06; A61P 35/00; C07K 16/28; G01N 33/574

(52) U.S. Cl. CPC A61K 31/58; A61K 31/4152; A61K 31/568; A61K 38/09; A61K 45/06; A61P 35/00; C07K 16/2818; C07K 16/2827; G01N 33/57434; G01N 2800/52

(57) ABSTRACT

Disclosed are methods for treating men with castrate resistant prostate cancer with a sufficient dose of testosterone to achieve supraphysiologic serum levels of testosterone in sequence with androgen ablative treatment or androgen synthesis inhibitors for one or more cycles until evidence of radiographic progression, at which point the subject will receive immune checkpoint blockade therapy.

### RELATED APPLICATION INFORMATION

This application is a national stage application under 35 U.S.C. § 371 of PCT/US2022/026376, filed Apr. 26, 2022, which claims priority to U.S. Application No. 63/179,709 filed on Apr. 26, 2021, the contents of which are herein incorporated by reference.

### STATEMENT OF GOVERNMENTAL INTEREST

This invention was made with government support under W81XWH-14-2-0189 awarded by the Department of Defense. The government has certain rights in the invention.

### BACKGROUND

Prostate cancer is uniformly lethal once it has escaped the confines of the prostate gland, resulting in the death of more than 30,000 American men each year. Jemal et al., 2008. Androgen ablation therapy has remained the standard of care for men with recurrent/metastatic cancer since its discovery by Charles Huggins in the 1940s. Huggins and Hodges, 1941. While androgen ablation therapy provides significant palliative benefit, all men undergoing androgen ablation eventually relapse and no longer respond to androgen ablation no matter how completely given. Crawford et al., 1989; Laufer et al., 2000. This observation led to the labeling of patients progressing on androgen ablative therapies as having “androgen independent” or “hormone refractory” prostate cancer.

New findings, however, have demonstrated that the majority of prostate cancer specimens from androgen ablated patients continue to express the androgen receptor (AR), often at higher levels. Linja et al., 2001; Shah et al., 2004. In addition, variants of AR that do not bind to ligand also are upregulated in androgen deprived prostate cancer cells. Prostate cancer cells from these castration refractory patients continue to express AR-regulated genes, such as prostate-specific antigen (PSA). This observation has resulted in a reclassification of “hormone refractory” disease as “Castration Resistant Prostate Cancer” (CRPC) and has opened up new avenues of research into the function of the AR in the androgen-deprived state.

These findings suggest that “castration-resistant” prostate cancer may continue to survive through aberrant AR signaling. Studies have demonstrated adaptation to chronic androgen deprivation through several mechanisms, including marked upregulation of the full-length AR, AR gene amplification, and expression of AR splice variants lacking the ligand binding domain. Shah et al., 2004; Linja et al., 2001; Chen et al., 2004; Isaacs et al., 2012. This observation has led to a renewed interest in the AR axis as a therapeutic target. On this basis, enzalutamide, a new antiandrogen, and abiraterone acetate, a CYP17 androgen synthesis inhibitor, have recently been approved as second line therapy for prostate cancer on the basis of modest observed improvements in overall survival versus placebo in randomized phase III trials. de Bono et al., 2011; Scher et al., 2012; Ryan et al., 2013; Beer et al., 2014.

A major mechanism for the development of CRPC following chronic exposure to androgen ablative therapies is the ability of prostate cancer cells to adapt to the lack of ligand by marked upregulation of the full-length AR and AR splice variants lacking the ligand binding domain. AR gene amplification also is commonly seen in samples from patients on chronic androgen deprivation. Laboratory studies have documented this upregulation of AR. These studies have demonstrated that this upregulation of AR may be responsible for the resistance to antiandrogens. In these studies, re-exposure of androgen-starved prostate cancer cells readapt upon exposure to androgen by lowering AR expression. This lowered AR expression now re-sensitizes these cells to androgen ablative therapies, such as antiandrogens. Chen et al., 2004; Isaacs et al., 2012.

In this background of renewed interest in blocking the AR, there has been the paradoxical observation that the growth of both androgen-sensitive and androgen-resistant prostate cancer cell lines is inhibited by the addition of testosterone or other synthetic androgens to the media. Isaacs et al., 2012; Umekita et al., 1996; Litvinov et al., 2006; Vander Griend et al., 2007. Typically, in vitro data in human prostate cancer cell lines demonstrate a biphasic response to androgens, with very low levels producing modest growth stimulation and expression of prostate tissue differentiation markers, such as PSA, while higher levels of androgen in the media suppress growth and PSA production. Isaacs et al., 2012; Umekita et al., 1996; Litvinov et al., 2006; Vander Griend et al., 2007. High levels in this case can be as low as picomolar concentrations suggesting that these “androgen ablation resistant” cells are exquisitely sensitive to androgens. These in vitro studies are supported by animal studies that have demonstrated that androgen receptor positive human prostate cancer cells selected to grow in castrated animals upregulate androgen receptor levels. Similar to the in vitro response, in these models, systemic testosterone administration produces significant growth inhibition, whereas antiandrogens, such as bicalutamide, promote prostate cancer growth.

Until recently, the mechanisms underlying this paradoxical response have been unknown. Recent data, however, have described several possible mechanisms for this effect of androgens on the growth of CRPC cells. The androgen receptor has been shown to be a licensing factor involved in DNA relicensing during progression through the cell cycle. Beer et al., 2014; Litvinov et al., 2006. AR is degraded as the prostate cancer cell goes through cycle. It has been demonstrated that the high levels of AR seen in CRPC cells do not get sufficiently degraded in the presence of high dose androgen due to androgen stabilization of the AR. Thus, under these conditions, AR remains bound to origins of replication preventing the cell from progressing through subsequent cell cycles and ultimately resulting in cell death. In addition, it has been demonstrated that replenishment of androgen to androgen-starved prostate cancer cells rapidly produces significant double strand DNA breakage that can result in inhibition of growth, inhibition of protein synthesis, growth and loss of clonogenic survival. Haffner et al., 2010. Finally, androgen-starved cells upregulate constitutively active AR splice variants that cannot bind androgen due to loss of the ligand binding domain. Dehm et al., 2008; Hu et al., 2009. CRPC cells may rely on these truncated AR variants for survival under low ligand conditions. It has been shown, however, that when androgen-starved CRPC cells are given high dose androgen, expression of these variants is rapidly downregulated to often undetectable levels. Hu et al, 2012; Thelen et al., 2013.

### SUMMARY

In some aspects, the presently disclosed subject matter provides method for treating prostate cancer in a subject in need of treatment thereof, the method comprising:
(a) administering to the subject a first dose of an androgen, or a derivative thereof, at a beginning of a first treatment cycle and a second dose, and optionally a third dose, of an androgen, or a derivative thereof, at a predetermined interval during the first treatment cycle, or if the third dose is administered, at a predetermined interval during a second treatment cycle; and
(b) sequentially administering one or more antiandrogens, one or more androgen synthesis inhibitors, or a combination thereof, at a completion of the first treatment cycle and throughout the second treatment cycle, and optionally throughout a third treatment cycle.

In certain aspects, the second dose, or optionally the third dose, of an androgen, or a derivative thereof, is administered about 28±5 days after the beginning of the first treatment cycle or, if the third dose is administered, about 28±5 days after the beginning of the second treatment cycle.

In more certain aspects, the method further comprises starting administration of the one or more antiandrogens, one or more androgen synthesis inhibitors, or a combination thereof, at the end of a one-, two-, or three-month androgen treatment cycle.

In certain aspects, the method comprises administering the one or more antiandrogens, one or more androgen synthesis inhibitors, or a combination thereof, each for one, two, or three 28±5 day treatment cycles.

In more certain aspects, the method further comprises discontinuing administration of the one or more antiandrogens, one or more androgen synthesis inhibitors, or a combination thereof, at a completion of the second treatment cycle, or optionally at a completion of the third treatment cycle, and restarting the first treatment cycle comprising administering to the subject a first dose of an androgen, or a derivative thereof, at a beginning of a first treatment cycle and a second dose, and optionally a third dose, of an androgen, or a derivative thereof, at a predetermined interval during the first treatment cycle, or if the third dose is administered, at a predetermined interval during a second treatment cycle.

In certain aspects, the method comprises alternating the first treatment cycle and the second treatment cycle, and optionally the third treatment cycle, until a clinical and/or radiographic progression is observed.

In other aspects, the presently disclosed subject matter provides a method for treating prostate cancer in a subject in need of treatment thereof, the method comprising:
(a) administering to the subject a first dose of an androgen, or a derivative thereof, at a beginning of a first treatment cycle;
(b) measuring a prostate-specific antigen (PSA) level of the subject; and
(c) one of:
   (i) maintaining a subject exhibiting a declining PSA level or no PSA progression on the first treatment cycle until PSA progression is observed; or
   (ii) discontinuing the first treatment cycle in a subject exhibiting a PSA progression (≥25% increase in PSA from baseline) and starting a sequential treatment cycle comprising administering to the subject one or more antiandrogens, one or more androgen synthesis inhibitors, or a combination thereof.

In certain aspects, the method further comprises continuing administration of the one or more antiandrogens, one or more androgen synthesis inhibitors, or a combination thereof, until PSA progression is observed.

In more certain aspects, the method further comprises discontinuing administration of the one or more antiandrogens, one or more androgen synthesis inhibitors, or a combination thereof, once the subject exhibits PSA progression (≥25% increase in PSA from baseline) and restarting the first treatment cycle.

In yet more certain aspects, the method further comprises alternating between the first treatment cycle and the second treatment cycle, and optionally the third treatment cycle, with onset of PSA progression until clinical and/or radiographic progression is observed.

In particular aspects of the presently disclosed methods, the first dose of an androgen, or a derivative thereof, and the second dose of an androgen, or a derivative thereof, and optionally the third dose of an androgen, or a derivative thereof, are each sufficient to achieve a supraphysiological serum concentration of testosterone in the subject.

In certain aspects, the supraphysiological serum concentration of testosterone in the subject is between about 3 to about 10 times a normal serum concentration of testosterone.

In particular aspects, the serum concentration of testosterone is greater than about 1,500 ng/dL.

In certain aspects of the presently disclosed methods, the androgen, or derivative thereof, is testosterone cypionate or testosterone enanthate at a dose of about 400 to about 500 mg.

In certain aspects of the presently disclosed methods, one or more androgens having a different biological potency are administered to the subject, wherein the first dose of an androgen, or a derivative thereof, and the second dose of an androgen, or a derivative thereof, and optionally a third dose of an androgen, or a derivative thereof, are given at a dose range that achieves a same relative supraphysiologic potency as that achieved with testosterone cypionate or testosterone enanthate a dose of about 400 mg to about 500 mg.

In certain aspects of the presently disclosed methods, the method comprises administering the androgen, or a derivative thereof, orally, transdermally or by intramuscular injection.

In particular aspects, the androgen, or a derivative thereof, comprises an ester of testosterone or an ester of dihydrotestosterone.

In certain aspects, the ester of testosterone of the ester of dihydrotestosterone is selected from a cypionate, enanthate, propionate, butyrate, and undecanoate ester of testosterone or dihydrotestosterone.

In particular aspects, the androgen, or derivative thereof, is testosterone cypionate or testosterone enanthate.

In certain aspects of the presently disclosed methods, the one or more antiandrogens is selected from the group consisting of bicalutamide, flutamide, nilutamide, apalutamide, darolutamide, enzalutamide, cyproterone acetate, proxalutamide, cimetidine, and topilutamide.

In particular aspects, the one or more antiandrogens is selected from the group consisting of enzalutamide, apalutamide, darolutamide, and combinations thereof.

In yet more particular aspects, the one or more antiandrogens is enzalutamide.

In certain aspects of the presently disclosed methods, the one or more androgen synthesis inhibitors is selected from the group consisting of a CYP17A1 inhibitor, a CYP11A1 (P450scc) inhibitor, a 5α-Reductase inhibitor, and combinations thereof.

In particular aspects, the one or more androgen synthesis inhibitors is selected from the group consisting of abiraterone acetate, ketoconazole, seviteronel, aminoglutethimide, alfatradiol, dutasteride, epristeride, finasteride, and combinations thereof.

In more particular aspects, the one or more androgen synthesis inhibitors is abiraterone acetate.

In certain aspects of the presently disclosed methods, the one or more antiandrogens, one or more androgen synthesis inhibitors, or a combination thereof, are administered at a dosage having a range selected from the group between about 100 to about 200 mg/day, between about 110 to about 190 mg/day, between about 120 to about 180 mg/day, between about 130 to about 170 mg/day, and about 160 mg per day.

In certain aspects of the presently disclosed methods, the method further comprises concurrently administering an androgen deprivation therapy (ADT) to the subject.

In particular aspects, the ADT comprises surgical castration or administering a luteinizing hormone-releasing hormone (LHRH) agonist or a LHRH antagonist to the subject.

In more particular aspects, the LHRH agonist is selected from the group consisting of leuprolide, goserelin, triptorelin, and histrelin.

In yet more particular aspects, the LHRH antagonist is selected from the group consisting of degarelix and relugolix.

In certain aspects of the presently disclosed methods, the method further comprises administering immune checkpoint blockade therapy to the subject if the subject exhibits clinical and/or radiographic progression.

In particular aspects, the immune checkpoint blockade therapy comprises administering an anti-PD1/PDL1 antibody or an anti-CTLA4 antibody.

In more particular aspects, the anti-PD1/PDL1 antibody is selected from the group consisting of pembrolizumab, nivolumab, and atezolizumab.

In more particular aspects, the anti-CTLA4 antibody comprises ipilimumab.

In certain aspects of the presently disclosed methods, the subject has progressive prostate cancer after treatment with abiraterone in combination with androgen deprivation therapy (ADT) as an initial therapy or as a second-line therapy after development of resistance to primary ADT.

In certain aspects of the presently disclosed methods, the prostate cancer comprises castration resistant metastatic prostate cancer.

In certain aspects of the presently disclosed methods, the subject is asymptomatic.

In other aspects of the presently disclosed methods, the subject is symptomatic.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one figure executed in color. Copies of this patent or patent application publication with color figures will be provided by the Office upon request and payment of the necessary fee.

Having thus described the presently disclosed subject matter in general terms, reference will now be made to the accompanying Figures, which are not necessarily drawn to scale, and wherein:

FIG. 1A and FIG. 1B show (FIG. 1A) Effect of synthetic androgen R1881 or bicalutamide on growth of ADT-resistant LNCaP and VCAP; (FIG. 1B) expression of full-length AR (AR-FL), AR-variants (AR-Vs) using antibody to AR N-terminal domain and AR-V& using AR-V7 specific antibody in the absence or presence of R1881;

FIG. 2 shows androgen-induced double strand DNA breaks in prostate cancer cell lines stimulated with high levels of androgen. Stimulation of androgen-deprived LAPC4 cells (control) to high levels of DHT (DHT) leads to numerous double strand breaks throughout the nucleus as evidenced by the accumulation of numerous γH2A.x foci, a marker for formation of double strand breaks;

FIG. 3A and FIG. 3B show castrated NOG mice inoculated with LNCaP/A-cells were either exposed to BAT therapy (via an implanted testosterone filled capsule that was placed and removed at two-week intervals) or left in a permanently castrate state (diamond versus box). (FIG. 3A) Evaluation of indicated parameters in LNCaP/A-cells growing in castrate mice vs. castrate mice supplemented with subcutaneous testosterone-filled silastic implants; (FIG. 3B) Immunohistochemical staining for AR in harvested LNCaP/A-xenografts growing in castrate vs. castrate+T-pelleted mice. Blue arrows indicate mitotic figures;

FIG. 4A, FIG. 4B, and FIG. 4C show representative results from a clinical trial of BAT plus etoposide. (FIG. 4A) Schematic of study design; (FIG. 4B) Baseline characteristics of patients on study; and (FIG. 4C) Mean serum testosterone levels at indicated time points for patients on study;

FIG. 5A, FIG. 5B, FIG. 5C, and FIG. 5D show representative results from the pilot BAT study in men with CRPC. (FIG. 5A) Waterfall plot of best PSA response with 7/14 men and 4/14 men achieving PSA declines >30% and >50%, respectively (FIG. 5B) Representative patterns of PSA response in pilot study PSA and serum testosterone level in non-responder; (FIG. 5C) PSA response in patient receiving 3 cycles of T+E and then 13 additional cycles of testosterone alone. Patient demonstrated renewed sensitivity to ADT after progressing on BAT; and (FIG. 5D) Objective lymph node complete and partial response after 3 cycles of BAT+E;

FIG. 6A, FIG. 6B, FIG. 6C, and FIG. 6D are representative clinical data from the RESTORE Study. (FIG. 6A) Trial design; (FIG. 6B) Response parameters for patients progressing on enzalutamide (Cohort A) or abiraterone (Cohort B); PSA50 to re-challenge post-BAT with enzalutamide was 71% vs 21% for abiraterone; (FIG. 6C) Adverse Events due to BAT in at least 10% of patients; and (FIG. 6D) Results of QoL surveys comparing 12 weeks of BAT vs. baseline on ADT alone;

FIG. 7A, FIG. 7B, FIG. 7C, FIG. 7D, and FIG. 7E show representative clinical data from the TRANSFORMER Study. (FIG. 7A) Trial design; (FIG. 7B) Response parameters for BAT (Arm A) vs. enzalutamide (Arm B) and results from patients who crossed over to opposite treatment; (FIG. 7C, FIG. 7D) Waterfall plots of PSA response showing initial PSA50 of 26.4% for BAT and 25.5% for enzalutamide and 72.7% in patients crossing from BAT to enzalutamide and 22.2 for patients crossing from enzalutamide to BAT; and (FIG. 7E) Kaplan-Meier curve of sum of PSA progression in each stage of trial. (PSA PFS2) demonstrates 2-fold increase in survival for BAT-enzalutamide (28.2 months) vs. enzalutamide-BAT (14.2 months) (HR=0.45, p=0.03);

FIG. 8A, FIG. 8B, and FIG. 8C show PSA levels in subjects administered immune checkpoint blockade therapy;

FIG. 9A, FIG. 9B, FIG. 9C, FIG. 9D, FIG. 9E, and FIG. 9F demonstrate that SupraT induces ferritinophagy in PCa cells. FIG. 9A, Western blot analysis showing ferritin and loading control vinculin in LNCaP and LAPC4 cells treated with indicated concentrations (nM) of R1881 (T) and vehicle (C) for 72 h. A representative of three independent experiments. Ferritin ˜21 kDa; loading control Vinculin 124 kDa. Bar graph shows mean relative density from three experiments. FIG. 9B, A representative single confocal section from three independent experiments is shown. Each experiment had at least 5 random fields LNCaP and LAPC4 cells were stained for LC3B (green) and ferritin (red). Right most panels show enlarged inset images of a region of interest, and the arrow-head indicates co-localization of ferritin in autophagosomes (LC3B). FIG. 9C, Western blot for immunoprecipitated NCOA4-FLAG for interaction with ferritin in LNCaP cells transfected with human NCOA4α-pHAGE-C-FLAG-HA plasmid. Non-transfected (C) and NCOA4α-pHAGE-C-FLAG-HA transfected vehicle treated cells (C*) were used as controls. A representative of three independent experiments. NCOA4 70 kDa and Ferritin ˜21 kDa.

FIG. 9D, Ferritin and LC3B levels probed by western blot in vehicle (C) or R1881 (T) treated LNCaP or NCOA4 shRNA lentiviral particle transduced LNCaP sh^{NCOA4 (1-4)} cells. A representative of three independent experiments. Ferritin ˜21 kDa; LC3-B 14, 16 kDa; loading control Vinculin 124 kDa. FIG. 9E, Cell death in LNCaP and LNCaP sh^{NCOA4 (1-4)} measured using trypan blue exclusion assay, Control (C), 10 nM R1881 (T). Bar graph shows mean % cell death from three independent experiments and error bar shows standard deviation. FIG. 9F, Western blot analysis for ferritin LNCaP cells treated with vehicle (C), 10 nM R1881 (T), 10 μM hydroxychloroquine (HCQ), 10 μM MG132 (MG132) alone or combination of T with either of HCQ or MG132. A representative of three independent experiments. Ferritin ˜21 kDa; loading control Vinculin 124 kDa. Scale bars: 15 μm (FIG. 9B);

FIG. 10A, FIG. 10B, FIG. 10C, FIG. 10D, FIG. 10E, and FIG. 10F demonstrate that SupraT induces ferroptosis in PCa cells. FIG. 10A, A representative fluorescence microscopy images of control (C) and 10 nM R1881 treated (T) LNCaP and LAPC4 cells from three independent experiments showing C11-BODIPY staining for oxidized membrane lipid (green fluorescence). Each experiment had at least 5 random field images. Enlarged inset images in the rightmost panels show a region of interest. FIG. 10B, Bar graph showing fold change in levels of control normalized oxidized lipids (Bodipy C11) in LNCaP and LAPC4 cells, measured by image analysis using ImageJ. FIG. 10C, Measurement of labile iron pool (Fe2⁺) in LNCaP and LAPC4 cells. Bar graph shows fold change in labile iron pool calculated by normalizing to untreated control cells. FIG. 10D, Quantitative RT-PCR analysis for a panel of pro-ferroptotic genes in 10 nM R1881 treated LNCaP, and LAPC4 cells post 72 h. Vehicle control was used for normalization to calculate the fold change. A representative of three independent experiments. Bar graph indicates fold change upon SupraT treatment. FIG. 10E, Cell death measured using trypan blue exclusion assay. Control (C), 10 nM R1881 (T), 10 μM Ferrostatin (F), or combination (T+F) shown as mean from three measurements with error bar showing standard deviation. FIG. 10F, Bar graph showing the mean of the number of colonies from three independent measurements counted using Fiji software for treatment conditions stated in (E) with error bars showing standard deviation. Asterisk * compares T or F vs. C and # indicates comparison between T and T+F. Both * and # indicates statistically significant differences (p<0.05). Scale bars: 100 μm (FIG. 10A);

FIG. 11A, FIG. 11B, FIG. 11C, FIG. 11D, FIG. 11E, FIG. 11F, and FIG. 11G demonstrate that SupraT induces nucleophagic degradation of unrepaired damaged DNA. FIG. 11A, A representative single confocal section is shown from three independent experiments. LNCaP cells stained for γ-H2AX (red) and LC3B (green) after treatment with 1 and 5 nM R1881. Arrow-heads indicate cells harboring γ-H2AX foci with lower LC3B puncta. Each experiment had at least 5 random field images. FIG. 11B, Scatter plot showing a correlation between the number of LC3B puncta and the number of γ-H2AX foci counted (n=19 measurements). Counting of puncta was performed using the image analysis software Fiji. FIG. 11C, Confocal microscopy images for PCa cell lines treated with 10 nM R1881 (T) and hydroxychloroquine (HCQ). Each experiment had at least 5 random field images. The lower panel is an inverted and magnified image of a single cell in the view-field for better visualization. Arrows indicate the localization of cytoplasmic DNA. FIG. 11D, Confocal images showing mitochondrial staining in LNCaP cells treated with vehicle (C), 10 nM R1881 (T), 10 μM HCQ, or combination of T+HCQ for 72 h. Lower panel shows a magnified view for enhanced visualization. Each experiment had at least 5 random field images. FIG. 11E, Photomicrographs showing colocalization of LC3B (green) and DAPI (Gray) in LNCaP cells treated with control (C), 10 nM R1881 (T), HCQ (10 μM) or T+HCQ treated LNCaP cells (72 h). Rightmost panel shows colocalized pixels. FIG. 11F, Graphical representation of fluorescence intensities of LC3B and DAPI on individual autophagosomes (n=16 measurements). FIG. 11G, Photomicrographs showing LC3B (green) and γ-H2AX (red) in LNCaP cells after 72 h of treatment with T+HCQ. Lower panels show the enlarged insets with a region of interest. Arrow-heads in the lower-left image indicate the presence of DNA (DAPI) in autophagosomes (LC3B), and lower-right image show γ-H2AX positivity in DNA present in those autophagosomes. Each experiment had at least 5 random field images. Scale bars: 5 μm (FIG. 11A), 10 μm (FIG. 11C), 10 μm (FIG. 11D), 10 μm (FIG. 11E), and 10 μm (FIG. 11G);

FIG. 12A, FIG. 12B, FIG. 12C, FIG. 12D, FIG. 12E, FIG. 12F, FIG. 12G, FIG. 12H, FIG. 12I, and FIG. 12J demonstrate that SupraT activates cytoplasmic nucleic acid sensors and subsequent NF-kappaB signaling. FIG. 12A, Western blot analysis showing DNA (STING) and RNA (RIG-I and MDA5) specific sensors along with actin as a loading control in LNCaP and LAPC4 cells treated with vehicle control (C) or 10 nM R1881 (T) in a time-dependent manner. A representative of three independent experiments. STING 33-35 kDa; RIG-I 102 kDa; MDA5 135 kDa; loading control Actin 43 kDa. FIG. 12B, Immunoblotting for LC3B and STING on fractions isolated using sucrose gradient centrifugation from LNCaP cell homogenates after 72 h of 10 nM R1881 treatment with 10 nM R1881 (T) or vehicle control (C). A representative of at least three independent experiments. STING 33-35 kDa; LC3B 14, 16 kDa. FIG. 12C-FIG. 12D, Native western blot probing for STING (FIG. 12C), and MAVS (FIG. 12D) in PCa cell lines treated vehicle (C) or 10 nM R1881 (T). A representative of three independent experiments; Loading control Actin 43 kDa. PC3 cells with constitutive expression of active STING were used as a positive control (C). FIG. 12E-FIG. 12F, Western blot analysis for AIM2 induction (AIM2 (FIG. 12E)) and signaling (IL-1β (FIG. 12F)) in PCa cell lines treated with vehicle (C) or 10 nM R1881 (T) in a time-dependent manner. A representative of three independent experiments. AIM2 40 kDa; IL-1β 17.31 kDa; Cleaved IL-1ß 17 kDa; loading control Vinculin 124 kDa. FIG. 12G-FIG. 12H Immunoblotting for interferon regulatory genes (IRF7, IRF3, TBK1 (FIG. 12G), and NF-kappaB (FIG. 12H) in PCa cell lines treated with vehicle (C) or 10 nM R1881 (T) at 24 h, 48 h, and 72 h. Activated forms of all interferon regulatory genes were probed by respective phosphorylation specific antibodies. A representative of at least two independent experiments. Phospho-TBK1 84 kDa; TBK1 84 kDa; Phospho IRF3 45-55 kDa; IRF3 50-55 kDa, Phospho-IRF7 65 kDa; IRF7 65 kDa; loading control Actin 43 kDa. FIG. 12I, Nuclear localization of IRF-7 and NF-kappaB in LNCaP cells after treatment with vehicle (C) or 10 nM R1881 (T) at indicated time points. A representative of at least two independent experiments. IRF7 65 kDa; NF-KappaB 65 kDa, loading control Histone H3 17 kDa. FIG. 12J, Bar graphs showing mean of luciferase NF-kappaB activity in LNCaP cells after treatment with vehicle (C) or 10 nM R1881 (T) using luciferase-based NF-kappaB reporter assay from three independent measurements and error bars show standard deviation. An asterisk indicates statistically significant differences (p<0.05);

FIG. 13A, FIG. 13B, FIG. 13C, FIG. 13D, FIG. 13E, FIG. 13F, FIG. 13G, and FIG. 13H demonstrate that SupraT induced NF-kappaB signaling drives innate immune response. FIG. 13A, Immunoblotting for NIK, p100, p52, and RELB proteins in vehicle control (C) or 10 nM R1881 treated (T) LNCaP cells. A representative of at least three independent experiments. NIK 125 kDa; NF-kappaB 2 p100 and p52 120 and 52 kDa respectively; RELB 70 kDa; loading control Actin 43 kDa. FIG. 13B, Immunofluorescence images for the LNCaP and LAPC4 cells treated with vehicle (C) or 10 nM R1881 (T) and stained with p52 (green) and DAPI (blue) for visualization of nuclear p52 protein. A representative of at least two independent experiments. Each experiment had at least 5 random field images. FIG. 13C, Quantitative RT-PCR for the interferon related genes in 10 nM R1881 treated LNCaP and LAPC4 cells for 24 h, 48 h, and 72 h time points. Mean fold change data between LNCaP and LAPC4 cells were compared statistically, and an asterisk indicates a statistically significant difference (n=3). Bar graph indicates fold change upon SupraT treatment. FIG. 13D, CXCL10 transcript analysis using quantitative RT-PCR from total RNA isolated from vehicle or 10 nM R1881 treated LNCaP and LAPC4 cells (n=3). Bar graph indicates fold change upon SupraT treatment. FIG. 13E, Western blot for CXCL10 protein (lysates for cellular and culture media for secreted) for vehicle (C) and 10 nM R1881 (T) treated LNCaP and LAPC4 cells made after 24 h, 48 h, or 72 h post-treatment (band marked with asterisk*). A representative of at least two independent experiments. CXCL10 10 kDa; loading control Actin 43 kDa. FIG. 13F, RT-PCR based measurement for CXCL10 transcripts in LNCaP or knockouts of RIG-I, STING, or both after treatment with 10 nM R1881 for 72 h. Bar graph indicates mean fold change upon SupraT treatment (n=3) and an asterisk indicates a statistically significant difference (p<0.05). FIG. 13G, Histogram showing endogenous retroviral transcripts in 10 nM R1881 treated LNCaP or LAPC4 cells. Bar indicates a mean fold change upon SupraT treatment (n=3), and the error bar indicates the standard deviation from three replicate values. FIG. 13H, Expression of CXCL10 transcripts in wild type, p65 KO, RELB KO, and TBK1 KO LNCaP cells treated with 10 nM R1881. Statistical significance is calculated between the wild type and knockout variants of LNCaP cells. Bar graph indicates fold change upon SupraT treatment (n=3) and an asterisk indicates a statistically significant difference (p<0.05). Scale bars: 15 μm (FIG. 13B);

FIG. 14A, FIG. 14B, FIG. 14C, FIG. 14D, FIG. 14E, and FIG. 14F demonstrate that SupraT activates both canonical and non-canonical NF-kappaB signaling. FIG. 14A, Immunoblotting for p100, p52, and RELB protein to show non-canonical NF-kappaB signaling in wild type and knockout LNCaP cells treated with vehicle (C) or 10 nM R1881 (T). A representative of three independent experiments. p100 and p52 120 and 52 kDa respectively; RELB 70 kDa; loading control Actin 43 kDa. FIG. 14B, Blot depicting stabilization of NIK in wild type and all knockout cells treated with vehicle (C) or 10 nM R1881 (T). A representative of three independent experiments. NIK 125 kDa; loading control Actin 43 kDa. FIG. 14C, Levels of p100 and p52 protein in DNA and RNA sensors (RIG-I and STING) single and double knockouts in LNCaP cells treated with vehicle (C) or 10 nM R1881 (T). A representative of three independent experiments. NF-kappaB2 p100 and p52 120 and 52 kDa respectively; RELB 70 kDa; loading control Actin 43 kDa. FIG. 14D, Heat map depicting fold change for expression of a number of genes selected from pan-cancer immune profiling panel. Total RNA extracted from indicated samples was analyzed for 770 immune-related human genes using nCounter human PanCancer Immune Profiling Panel (Nanostring). FIG. 14E, Heatmap showing relative expression levels of genes selected from the list of 57 genes that showed higher expression in Nanostring analysis. Total RNA was analyzed using qRT-PCR, and data are shown as heatmap from a representative experiment of three independent experiments. FIG. 14F, Representative photomicrographs depict CFSE labeled NK-92 cells migrated towards either vehicle (C) or 10 nM R1881 (T) treated LNCaP or LAPC4 cells through 5 μM Boyden chamber. Vehicle control normalized count of NK-92 cells plotted as a mean of four independent experiments on histograms with error bars showing standard deviation. Each experiment had at least 5 random field images. An asterisk indicates a statistically significant difference (p<0.05) Scale bars: 100 μm (FIG. 14F); and

FIG. 15A, FIG. 15B, FIG. 15C, FIG. 15D, FIG. 15E, FIG. 15F, and FIG. 15G demonstrate that SupraT induces tumor infiltration of immune cells. FIG. 15A, Immunofluorescence staining of tumor xenograft specimens for LC3B (green) and CXCL10 (red) from tumor-bearing mice treated with testosterone cypionate (T) (n=4) in the lower panel as compared to tumor isolated from vehicle (C) treated animals (n=4) shown in the top panel. Each sample had at least 5 random field images. FIG. 15B, Magnified view of a tumor cell showing cytoplasmic localization of CXCL10 (red). Bar graph shows CXCL10 measured by analyzing images (at least five fields) on ImageJ in control and testosterone treated groups. FIG. 15C, Tumor xenografts specimens from testosterone cypionate treated mice stained with DAPI (grey) and LC3B (green) to show the presence of cytoplasmic autophagosomal DNA. At least 5 random images were collected for each sample. FIG. 15D, FIG. 15E, and FIG. 15F, Representative immunofluorescence images for tumor sections from the vehicle (C) (n=4) or testosterone cypionate treated (T) mice (n=4) stained with Alexafluor488-antiCD57 (FIG. 15D), Alexafluor488-antiF4/80 (FIG. 15E), and Alexafluor488-antiLy-6G (FIG. 15F). The number of stained NK cells, Macrophages and Neutrophils were counted using Fiji image analysis software and plotted as mean on histogram. Each sample had at least 5 random field images. FIG. 15G, Immunohistochemical staining for CD8 in matched biopsy before and during BAT treatment (n=10). The left photomicrograph shows a medium power image of tumor with sparse CD8 cell infiltrates involving tumor, most of the cells seen represent tumor cells. Note an increase in the extent of infiltrate in the treated sample in image on the right. Arrows indicate CD8 positive T cells. The histogram shows area normalized number of CD8 T cells in biopsies collected from PCa patients before and after BAT regimen (n=10). An asterisk indicates a statistically significant difference (p<0.05). Scale bars: 10 μm (FIG. 15A), 2 μm (FIG. 15B) 25 μm (FIG. 15C) 20 μm (FIG. 15D and FIG. 15E).

FIG. 16 shows a schematic providing the presently disclosed dosage regimen.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Androgen Ablative Therapy for Prostate Cancer

In the current regimen for treating castration resistant prostate cancer (CRPC), men with recurrent or metastatic prostate cancer are initially treated with LHRH agonists, which typically results in castrate levels of serum testosterone (i.e., <50 ng/dL) within 2-4 weeks post initiation of therapy. Noonan et al., 2013. The catastrophic loss of androgen as their major growth and survival factor results in the death of the majority of prostate cancer cells. On this basis, the majority (approximately 90%) of men have an initial beneficial palliative response to androgen deprivation therapy (ADT).

Relapse, however, occurs in all men treated with ADT. Over time, prostate cancer cells that survive the initial acute drop in serum androgen adapt to the chronic low androgen conditions by upregulating AR through overexpression, gene amplification and expression of truncated, transcriptionally active AR splice variants (AR-V) that lack the ligand binding domain. The first clinical manifestation of this adaptive increase in AR signaling is the renewed production of PSA. At this point the patient is considered to have CRPC. Typically, this patient would continue on ADT and begin second-line hormonal therapies. This approach is based on the concept that a sufficient amount of androgen is produced by the adrenal glands, and perhaps by the prostate cancer cells themselves, Haffner et al., 2010, to support the growth of the surviving adapted CRPC cells. Thus, second line hormonal therapies were developed that either competitively inhibit androgen binding to AR (e.g., antiandrogens flutamide, bicalutamide, and nilutamide) or inhibit adrenal androgen synthesis (e.g., ketoconazole). Noonan et al., 2013. While clinical benefit was demonstrated, until recently, the effect of second-line therapy on survival was unknown due to lack of appropriately powered studies. Enzalutamide and abiraterone, however, have both received FDA-approval for use in metastatic CRPC based on a modest survival benefit observed in large, randomized studies, Table 1. de Bono et al., 2011; Scher et al., 2012; Ryan et al., 2013; Beer et al., 2014.

The current treatment approach for CRPC is to continue chronic LHRH agonist therapy despite progression and administer “second line” hormone therapy. Based on a demonstrated survival benefit, abiraterone is emerging as the preferred initial second line therapy. Before the availability of enzalutamide, standard therapy in men with progression on abiraterone would be to give docetaxel chemotherapy. Currently, enzalutamide is FDA-approved for use in men post-docetaxel based on Phase III results showing approximately 5 months improvement in survival in the post-docetaxel setting, Table 1. Linja et al., 2001. Enzalutamide, however, is frequently being administered to men prior to docetaxel if insurance clearance can be obtained. It also is expected that enzalutamide, like abiraterone, will be approved for use in the pre-chemotherapy setting based on positive results from the PREVAIL study that showed an improvement in median overall survival that was estimated at 32.4 months in the enzalutamide group and 30.2 months in the placebo group (hazard ratio, 0.71; 95% CI, 0.60 to 0.84; P<0.001). Isaacs et al., 2012.

Thus, based on the result of these studies, the ease of administration of these oral agents, and the possibility of delaying the need for chemotherapy, the evolving treatment paradigm will likely involve the sequential addition of abiraterone and enzalutamide to LHRH agonist-based ADT in men with ADT progression. Several small studies, however, have demonstrated that sequential use of these agents in the post-chemotherapy setting is associated with significant reduction in time to radiographic progression to <5 months, decreased PSA response, and objective response suggesting cross-resistance between these agents, Table 1. Loriot et al., 2013; Noonan et al., 2013; Schrader et al., 2013; Bianchini et al., 2013. Limited information is available on progression-free survival with enzalutamide post-abiraterone in the pre-chemotherapy setting, but data on small number of patients indicates median rPFS≤6 months. Zhang et al., 2014; Suzman et al., 2014. The mechanisms underlying this reduced response rate are likely multi-factorial and may include continued adaptive increase in AR expression, increased expression of ligand independent AR variants in resistant cells and emergence of AR mutations that may affect enzalutamide binding.

TABLE 1
Results from sequential Treatment Enzaluatmide
after Abiraterone in CRPC. (15, 18-23)

| | | >50% | | | |
|---|---|---|---|---|---|
| | | decline | PSA | | Objective |
| | N= | PSA (%) | PFS | TTP (m) | Response (%) |
| Enzalutamide | 872 | 78 | 11.2 | 20 | 59 |
| Phase III | | | | | |
| (PREVAIL) | | | | | |
| Enzalutamide | 35 | 28.6 | NA | 4.9 | 2.9 |
| post | 39 | 12.8 | NA | 2.8 | 4.3 |
| Abiraterone | 79 | 18 | 4.0 | NA | NA |
| | 102 | 29 | 3.0 | NA | NA |
| | 30 | 34 | 4.1 | 4.7 | NA |
| | 47 | 25.5 | NA | 6.6 | NA |
| | 214 | 27 | 5.7 | 8.1 | NA |

### B. AR-Splice Variants in CRPC

The AR protein contains several functional domains. Hu et al., 1010. The N-terminal domain (NTD), encoded by exon 1, constitutes approximately 60% of the 110-kDa full-length protein and is the transcriptional regulatory region of the protein. The central DNA-binding domain (DBD) is encoded by exons 2 and 3, whereas exons 4 to 8 code for the highly conserved C-terminal ligand-binding domain (LBD) which is the intended target of all current existing AR-directed therapies. Hu et al., 2010.

Recently, AR variant (AR-Vs) transcripts that are encoded by aberrantly spliced AR mRNA have been discovered that lack the reading frames for the ligand-binding domain due to splicing of “intronic” cryptic exons to the upstream exons encoding the AR DNA-binding domain. Hu et al., 2010. Fifteen AR-Vs have been fully decoded, with the variant AR-V7 representing the single most important AR-V for which expression levels of its mRNA and protein can be detected in the vast majority of clinical CRPC specimens by variant-specific probes and antibodies. Hu et al., 2009; Hu et al., 2010.

To evaluate mRNA expression of full-length AR and AR-V7 in CTC, the Alere™ CTC AdnaTest, a commercial diagnostic test in CE-marked countries, was modified. The ProstateCancerSelect kit was used to enrich circulating prostate tumor cells in the blood using magnetic beads coated with three different antibodies (EPCAM and two proprietary antibodies). Cell capture by the optimized antibody combination is followed by cell lysis and RT-PCR analysis using a combination of multiple mRNA markers of the AR axis, using the ProstateCancerDetect kit and custom probes. Specific probes were developed to detect both the canonical AR-FL and AR-V7 that both increase upon treatment with enzalutamide and abiraterone in cell line and xenograft models. Hu et al., 2010. This test and methodology were used to interrogate CTCs for the presence or absence of AR-V7 from prospectively enrolled patients with metastatic castration-resistant prostate cancer initiating treatment with either enzalutamide or abiraterone. Antonarakis et al., 2014. Associations between AR-V7 status and PSA response rates, PSA-progression-free survival (PSA-PFS), and clinical/radiographic progression-free survival (PFS) were examined. Multivariable Cox regression analyses were performed to determine the independent effect of AR-V7 status on clinical outcomes. Antonarakis et al., 2014. Thirty-one enzalutamide-treated patients and thirty-one abiraterone-treated patients were enrolled, of which 38.7% and 19.4% had detectable AR-V7 from CTCs, respectively. Antonarakis et al., 2014. Among men receiving enzalutamide, AR-V7-positive patients had inferior PSA response rates (0% vs 52.6%, P=0.004), PSAPFS (median: 1.4 vs 5.9 months, P<0.001), and PFS (median: 2.1 vs 6.1 months, P<0.001) compared to AR-V7-negative patients. Similarly, among men receiving abiraterone, AR-V7-positive patients had inferior PSA response rates (0% vs 68.0%, P=0.004), PSA-PFS (median: 1.3 months vs not reached, P<0.001), and PFS (median: 2.3 months vs not reached, P<0.001). The negative prognostic impact of AR-V7 detection was confirmed in multivariable analyses. Antonarakis et al., 2014. The conclusion from this study is that the presence of AR-V7 in CTCs from patients with castration-resistant prostate cancer predicts resistance to enzalutamide and abiraterone. Antonarakis et al., 2014.

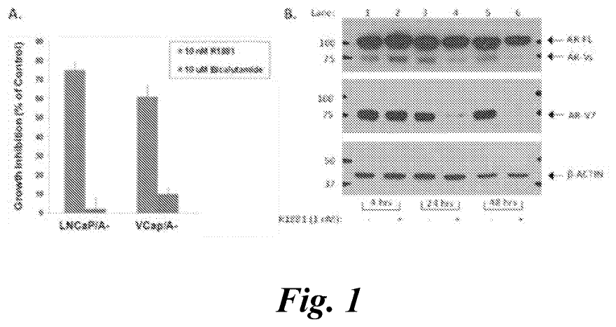

Recently, Hu et al. demonstrated that human CRPC cells LNCaP95 and VCaP rapidly downregulate expression of all AR isoforms following exposure to high dose androgen, Hu et al., 2012, suggesting a potential for resensitization to anti-androgens following exposure to high dose-T. Androgen exposure resulted in rapid decrease or loss of AR-V7 nuclear staining in these cell lines. These results were further confirmed by Thelen et al., who demonstrated that androgen treatment of the AR-V7 overexpressing human prostate cancer cell line VCaP results in rapid downregulation of total AR levels and almost complete loss of AR-V7 expression. Thelen et al., 2013. These AR-V expressing cells are profoundly growth inhibited by high dose levels of androgens, FIG. 1A. In contrast, these lines are highly resistant to the anti-androgen bicalutamide. Complete loss of AR-V expression in VCaP cells over a 48-hr exposure to the synthetic androgen R1881 also was observed, FIG. 1B.

On the basis of these pre-clinical and clinical results, the Alere™ CTC AdnaTest can be used to isolate blood samples from subjects undergoing the presently disclosed treatment protocol at screening, after three months on BAT or enzalutamide, and at time of radiographic progression to determine the effect of each treatment arm on the expression of full-length AR and AR-V7.

### C. Preliminary Related to Testosterone Therapy in CRPC

### C.1 Androgen Produces Double Strand Breaks in Human Prostate Cancer Cells

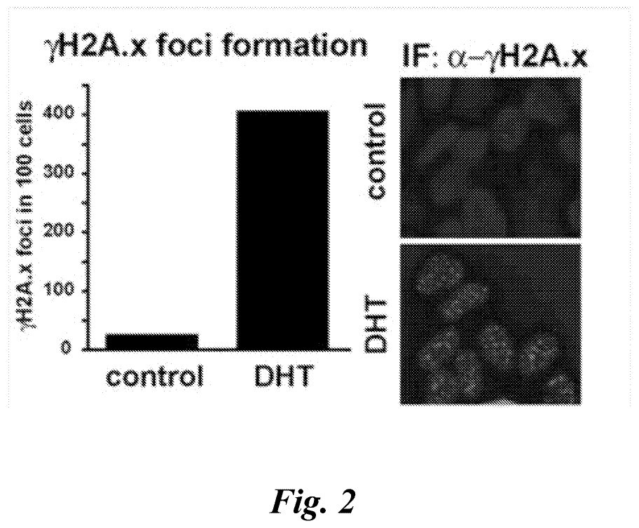

The mechanisms underlying the growth suppressive effects of high levels of androgens in prostate cancer cells in vitro and in vivo is likely highly complex. Recent evidence from suggests that one mechanism may involve the formation of androgen-induced Topoisomerase II beta (TOP2B) mediated double strand breaks at AR target genes, FIG. 2, Haffner et al., 2010. Recent studies have shown that estrogen signaling in breast cancer cells involves the co-recruitment of Estrogen receptor and TOP2B to estrogen receptor target sites, where TOP2B introduces transient double strand breaks.

Without wishing to be bound to any one particular theory, it is thought that such a mechanism may be involved in androgen signaling in prostate cancer cells and that at high doses of androgens, such breaks may persist and ultimately lead to growth suppression. In support of this hypothesis, it was observed that stimulation of androgen-deprived LNCaP cells with dihydrotestosterone (DHT) led to recruitment and catalytic activity of TOP2B at AR target sites in the TMPRSS2 enhancer, as well as at other known AR target sites. At high doses of DHT, this TOP2B recruitment and catalytic activity was associated with significant formation of AR and TOP2-dependent persistent double strand breaks at the TMPRSS2 gene, as observed by fluorescence in situ hybridization (FISH) assay capable of detecting genomic breaks on an individual cell basis. Haffner et al., 2010. Such breaks likely occurred throughout the genome at AR target sites since numerous γH2A.x foci, a marker for double strand break formation, were observed throughout the nucleus in response to stimulation of LNCaP cells with high-dose DHT (FIG. 2).

In further confirmation of this hypothesis, recruitment of ATM, a double strand break repair signaling protein, to AR target sites in PSA and TMPRSS2, genes present on different chromosomes in the cell, also was observed. These findings suggest that exposure of prostate cancer cells from patients with CRPC to high doses of testosterone may induce growth suppression due to the accumulation of androgen-mediated, TOP2-induced double strand DNA breaks. Haffner et al., 2010.

C.2 Adaptive Auto-Regulation of AR Leads to Over-Stabilization of the AR at Origins of Replication when Exposed to High Dose Androgen Levels

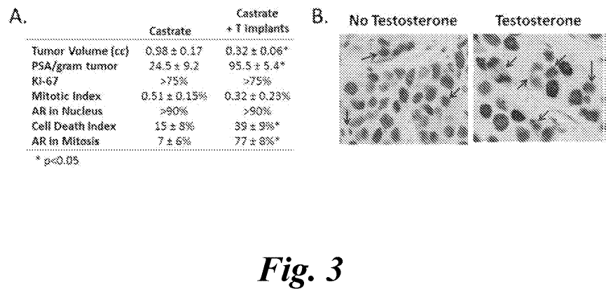

A mechanism for growth inhibition by testosterone in CRPC cells has been recently explored. Beer et al., 2014; Litvinov et al., 2006; Vander Griend et al., 2007. These studies document that the increase in AR expression observed in these cells in the low testosterone environment creates a unique therapeutic vulnerability to selectively kill CRPCs. This observation is based on the fact that AR is involved in DNA relicensing and DNA replication AR must degraded each cell cycle for proper relicensing to occur. Overstabilization of the increased levels of AR observed in CRPC with high dose testosterone prevents complete degradation of AR via the proteasome during mitosis. Beer et al., 2014; Litvinov et al., 2006; Vander Griend et al., 2007. This mechanism was demonstrated by in vivo treatment of resistant human LNCaP prostate cancer xenografts with testosterone implants to achieve high dose serum T-levels. Beer et al., 2014. This treatment resulted in significant growth inhibition, FIG. 3a. These growth inhibited cells had a similar amount of cells with nuclear AR in the nucleus, and Ki-67 positivity, FIG. 3a. In xenografts treated with high dose testosterone, however, the Cell Death Index was approximately 3-fold higher. Beer et al., 2014. More strikingly, the percent of cells staining positive for AR in mitosis was approximately 10-fold higher in cells exposed to high dose testosterone vs. castrate only animals, FIG. 3b.

These data suggest that CRPC cells that have not properly relicensed DNA can die when they attempt to proceed through a subsequent cell cycle. Thus, based on this proposed mechanism, prostate cancer cells that maintain high AR levels will be vulnerable to cell death when exposed to high dose testosterone conditions due to inability to rapidly auto-regulate AR to lower levels. Due to the bipolar cycling between high and low serum testosterone achieved with BAT, those cells that do manage to survive the high testosterone environment through adaptive down-regulation of AR will become vulnerable to cell death when suddenly exposed to low testosterone conditions that occur over the cycle of BAT.

D. Clinical Experience with Testosterone in Prostate Cancer

Until recently, there had been very limited clinical experience in the PSA-era treating CRPC patients with testosterone. Brendler et al. at the Brady Urological Institute reported in the Archives of Surgery in 1949 on the use of parenteral testosterone in several men with advanced CRPC. Brendler et al., 1949. They observed considerable improvement in several men that included decreased pain, decreased prostate size, and decreases in acid and alkaline phosphatase. In a second study, Prout and Brewer reported in Cancer in 1967 on the treatment of men who had been either untreated or recently castrated or long-term castrates with parenteral testosterone. Prout and Brewer, 1967. Five relapsed patients in the long-term castrate group received testosterone for at least one month and 4 of 5 had subjective improvement. Five remaining patients in the long-term castrate group received testosterone for 1-19 days, with each progressing and subsequently coming off therapy. Acid phosphatase declined in 2/5 men receiving a longer course of testosterone. Remarkably, one man in this group admitted to hospital with severe back pain, weakness and anorexia had a 10-month response with complete cessation of pain, excellent appetite and weight gain with decrease in acid phosphatase from 50 to 5 units.

In contrast, a number of studies during the 1960-70s evaluated the use of testosterone-priming in combination with ³²P-sodium phosphate to treat men with CRPC and severe pain due to widespread bony metastases. Donati et al., 1966; Johnson and Haynie, 1977. In these studies, initial testosterone-priming using a variety of parenteral dosing regimens was associated with transient increase in bone pain during the first week followed by excellent pain palliation following administration of ³²P. Similar results were observed in studies led by Manni, who evaluated testosterone-priming with chemotherapy in the 1980's. Manni et al., 1988. These studies also were conducted in men with CRPC and pain due to widely metastatic disease. In these studies, increased bone pain also was observed in men upon initial treatment with oral androgens. The increased pain in these studies typically occurred within days of testosterone administration. Thus, given this time frame, it is likely the increased pain was due to testosterone-stimulation of inflammation/cytokine release within sites of bone metastases rather than a direct effect on tumor growth. Such rapid change also is seen in men with bone pain upon initially starting ADT. Marked improvement in pain after ADT often occurs within hours of treatment, an effect not due to tumor death, but rather a rapid change in expression of pain-promoting gene products.

More recently, two Phase I studies were reported describing the results of the use of transdermal testosterone as therapy for men with CRPC who had minimal to moderate disease burden and no pain due to prostate cancer. In the first study, Szmulewitz et al. evaluated the effect of increasing doses of transdermal testosterone in 15 men with early CRPC (rising PSA and minimal bone disease). Szmulewitz et al., 2009. Five men each were treated with 2.5, 5.0 or 7.5 mg/day of transdermal testosterone, which brought the median concentration of testosterone from castrate to 305, 308 and 297 ng/dL respectively. In this study no grade 3 or 4 toxicities were observed with the exception of one man who was taken off study at week 53 for grade 4 cardiac toxicity. Only one patient had symptomatic progression and three patients (20%) had a decrease in PSA (largest was 43%). Patients treated at the highest testosterone dose had a prolonged time to progression that did not reach statistical significance, most likely due to the small cohort size. In the second study, Morris et al. evaluated the effect of transdermal testosterone at a dose of 7.5 mg/day administered for 1 week (n=3), 1 month (n=3) or until disease progression (n=6) in 12 patients with CRPC. Morris et al., 2009. They observed no grade 3 or 4 toxicities and no pain flares. Eugonadal serum testosterone levels were reported for this study. No objective responses were observed. Four patients had at least 20% declines and one achieved a >50% PSA decline.

Neither of these Phase I studies achieved the high dose levels of serum testosterone that can be reached with FDA-approved doses of testosterone administered as an intramuscular depot. Behre and Nieschlag, 2012. The levels of serum testosterone achieved, however, were in the high-end of the eugonadal range. Remarkably, although the studies were considered “negative” from the standpoint of disease response, in both studies the administration of parenteral testosterone to men with CRPC was very well-tolerated and did not result in significant worsening of disease or symptoms, including pain flares. While only one patient out of 27 from the combined studies had a reported >50% decline in PSA, smaller PSA declines were observed in a few of the patients on these two studies with a trend toward a dose-responsive effect, suggesting a potential for therapeutic benefit in some patients. Szmulewitz et al., 2009; Morris et al., 2009.

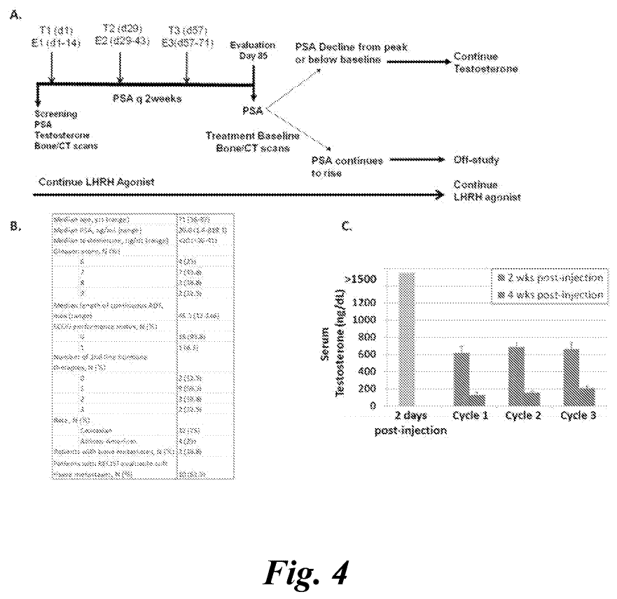

Based on the preclinical results and potential mechanisms for growth inhibition that include androgen-induced double strand breaks and stabilization of AR preventing relicensing, a pilot study was conducted to evaluate the efficacy and safety of pharmacologic doses of testosterone to produce high dose testosterone levels in conjunction with oral etoposide in chronically castrated men with rising PSA and CRPC, FIG. 4A. Patients who had been continuously castrate for more than one year with minimal metastatic disease burden (≤5 total bone metastases and ≤10 total sites of metastases) and/or rising PSA were eligible, FIG. 4B. To achieve rapid cycling between high dose and near castrate serum testosterone (i.e., BAT) patients received intramuscular injection of 400 mg testosterone cypionate every 28 days. For the first 3 cycles of therapy patients received BAT plus oral etoposide 100 mg po/day days 1-14 of a 28-day cycle. After 3 cycles PSA response and objective response were assessed. Those patients with a PSA that was declining from peak levels and no objective evidence of disease progression or worsening pain were continued on therapy. Given the toxicity associated with etoposide and the lack of clinical response in an earlier trial, Hussain et al., 1994, patients who responding after 3 cycles of testosterone plus etoposide were continued on testosterone alone based on protocol amendment.

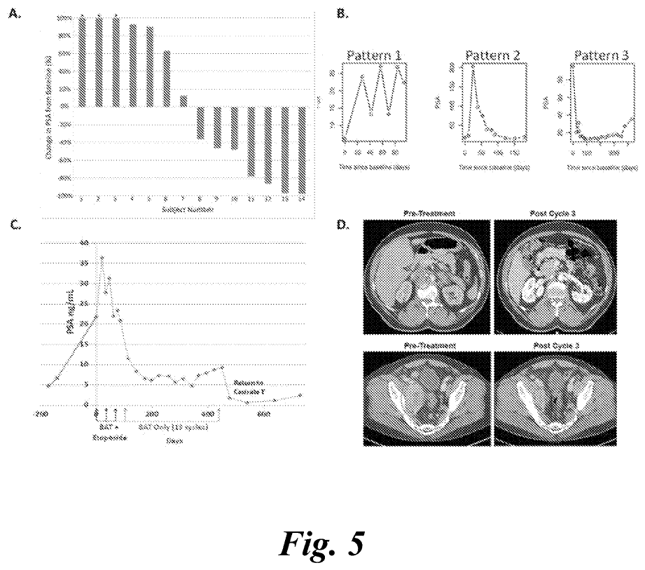

Seven of fourteen patients had a decline in PSA from baseline value, FIG. 5A. An eighth patient progressing after testosterone treatment for 6 months had a decline in PSA upon reaching castrate level testosterone. Non-responders came off of trial after 3 cycles due to PSA progression. Overall, three patterns of PSA response were observed, FIG. 5B. For the seven patients that had a PSA decline, the median time to PSA progression was 221 days (range, 95 to 454 days). The dose of 400 mg testosterone cypionate produces high dose levels >1500 ng/dL within 2 days post injection, FIG. 4C. At baseline, ten subjects had RECIST-evaluable soft tissue metastases, FIG. 4B. Of these patients, two (20%) had progressive disease (PD), three (30%) had stable disease after a median follow up of 91 days (range, 87 to 92 days), four (40%) had partial responses (PRs) and one (10%) had a complete response (CR), FIG. 5D. None of the 14 patients completing 3 months of therapy developed new bone metastases. One patient with >50% decrease in PSA had intensification of an isolated tibial metastases on bone scan and was removed from study despite decline in PSA levels. No other patient developed worsening pain on study.

### D.1 PSA Reductions to Subsequent Hormonal Therapies

As part of the aforementioned pilot study, a post hoc exploratory analysis on the effect of BAT on subsequent hormonal therapies was performed. Overall, 12 out of 13 subjects had PSA decline to AR-directed therapy post-BAT (one patient continued to show PSA progression upon return to castrate testosterone levels and proceeded to receive docetaxel, the 14th patient remains on BAT (Table 2). Testosterone levels were allowed to return to the castrate range prior to having a second line AR-directed therapy initiated (e.g., abiraterone, enzalutamide, and bicalutamide) in 12 patients. Of these 12 subjects, 9 (75%) had a PSA decline below their post-BAT PSA. Of the 6 PSA responders that came off study, 4 (66.7%) had a PSA decline below their post-BAT PSA upon becoming castrate again. All of the patients had received at least one anti-androgen prior to starting the study, Table 2. PSA response to secondary hormonal therapy after return to castrate T-levels post-BAT.

TABLE 2
PSA response to secondary hormonal therapy
after return to castrate T-levels post-BAT.

| | Maximum | | | Maximum PSA |
|---|---|---|---|---|
| | PSA decline | Secondary | Secondary | decline upon |
| | upon | HT | HT | secondary HT |
| Subject | recastration | received | received | initiation |
| Number^{a} | (%)^{b} | pre-study | post-study | (%)^{c} |
| 1 | NA | nilutamide, | nilutamide | −44.3 |
| | | bicalutamide | | |
| 2 | −88.2 | bicalutamide | none | NA |
| 3 | −57.4 | bicalutamide | abiraterone | −94.2 |
| 4 | no decline | bicalutamide | abiraterone | −92.7 |
| 5 | −52 | bicalutamide, | enzalutamide | −30.4 |
| | | ketoconazole | | |
| 6 | −58.2 | bicalutamide | bicalutamide | −30.8 |
| 7 | −73.2 | bicalutamide | abiraterone | −88.1 |
| 8 | no decline | bicalutamide, | none | NA |
| | | nilutamide | | |
| 9 | −92.5 | bicalutamide | none | NA |
| 10 | no decline | bicalutamide | abiraterone | −63.8 |
| 14 | −38.2 | nilutamide, | enzalutamide | −99.5 |
| | | bicalutamide, | | |
| | | abiraterone | | |
| 15 | −35 | abiraterone | enzalutamide | −78.3 |
| 16 | −68.4 | nilutamide, | enzalutamide | −53.2 |
| | | bicalutamide, | | |
| | | enzalutamide | | |

After return to castrate testosterone levels post-BAT, 10 of 10 (100%) patients receiving second line therapy with either abiraterone (n=4/4) or an anti-androgen [enzalutamide (n=4/4), bicalutamide (n=1/1), nilutamide (n=1/1)] had a PSA decline (range 30.8-99.5%) (FIG. 5). Four of 4 patients receiving abiraterone and 3/4 patients on enzalutamide had >50% PSA decline. Of note, two subjects were re-challenged with a first-generation anti-androgen (i.e., nilutamide, bicalutamide) and one with enzalutamide after having previously progressed on these agents. These subjects achieved a 44.3%, 30.9% and 53.2% PSA decline upon initiation of nilutamide, bicalutamide and enzalutamide, respectively. The patient re-challenged with enzalutamide also had previously progressed on abiraterone prior to enrolling in this study.

The lessons learned from this pilot trial were that high dose testosterone could be administered safely to men with metastatic CRPC without producing worsening signs or symptoms due to prostate cancer. While formal testing was not performed, most men reported improved quality of life with increased energy, less fatigue, increased libido and resumption of erectile function in those men with preserved function prior to castrating therapy. PSA decline/response and objective responses were observed in 50% patients who completed three cycles of therapy. While PSA declines were observed, many patients with supposedly CRPC had an initial spike in PSA following the first injection of testosterone. These results suggest that, although these patients demonstrated progressive disease while on chronic castrating therapies as a requirement for enrollment in the trial, the CRPC cells must have continued expression of a functional AR axis as evidenced by the increased expression of an androgen responsive gene, PSA, in response to androgen replacement. Finally, 100% of patients demonstrated PSA response to androgen ablative therapies post-BAT suggesting exposure to BAT has the potential to reverse resistance and re-sensitize CRPC cells to androgen ablative therapies, such as abiraterone and enzalutamide.

D.2 Further Clinical Studies of Supra Testosterone in Men with CRPC
D.2.1 Results from the-RE-Sensitizing with Supra Testosterone to Overcome Resistance (RESTORE) Study

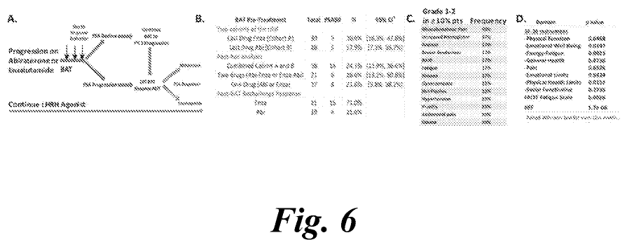

The RESTORE study (NCT02090114) was an NIH sponsored Phase II study designed to assess the safety and efficacy of BAT in men (n=30 per cohort) with CRPC progressing on enzalutamide (Cohort A) or abiraterone (Cohort B), FIG. 6A. Shah et al., 2004. The primary endpoint of the first part of the study was to assess PSA response to BAT. Secondary endpoints included objective response, safety, and quality of life. The second part of the study was to determine in BAT could re-sensitize patients to repeat exposure to second line therapy that patients were progressing on prior to receiving BAT. Thus, patients progressing on enzalutamide were re-exposed to enzalutamide after BAT and similarly for abiraterone. Both arms of this study met the primary endpoint. PSA50 Response to BAT was 30% in patient's post-enzalutamide and 18% post-abiraterone, FIG. 6B. Shah et al., 2004. PSA50 response was not significantly different in patients who had received two prior treatments (i.e., enzalutamide-abiraterone or abiraterone-enzalutamide) compared to only one. Overall duration of response to BAT and duration of response to re-exposure was longer in the post-enzalutamide vs. post-abiraterone cohort. The PSA50 response to re-exposure to enzalutamide was 71% vs. only 21% for patients re-exposed to abiraterone, FIG. 6B. Shah et al., 2004. A third cohort was later added (Cohort C) for men progressing on ADT alone. Overall, 90 men were treated on this study over a 4-year period (2014-2018).

Adverse events (AEs) to BAT were primarily Grade 1-2 with most common being generalized musculoskeletal pain and sexual side effects (breast tenderness, hot flashes and gynecomastia), FIG. 6C. Serious AEs occurred in individual patients and were not attributed to BAT with the exception of grade 3 hypertension that occurred in 3 patients. QoL surveys showed significant improvement in the FACIT Fatigue Scale, IIEF survey, Physical Function, Emotional Well-Being and Energy-Fatigue on the SF-36 Instrument subscales, for BAT compared to baseline on ADT, FIG. 6D.

D.2.2 Results from the TRANSFORMER Study

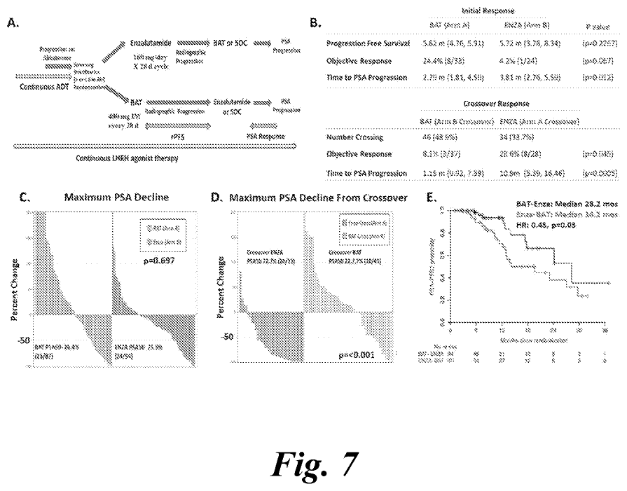

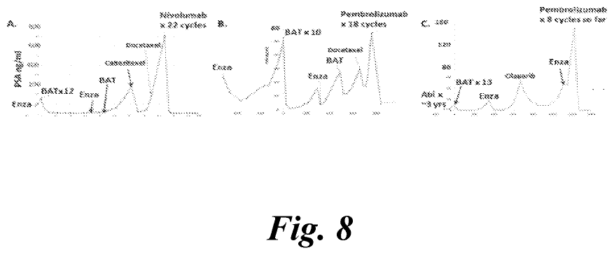

The TRANSFORMER study (NCT02286921) was a DOD-sponsored randomized Phase II study designed to compare the efficacy of BAT vs. enzalutamide in asymptomatic men with CRPC progressing on abiraterone, FIG. 7A. The study was conducted at 17 academic centers across the U.S. The study was coordinated by the Prostate Cancer Disease Group clinical research team at Johns Hopkins, who performed regulatory oversight, study/site monitoring, and data analyses. From 2015-2018, 195 patients underwent 1:1 randomization to either standard dose enzalutamide (n=101) or BAT (n=94) at 400 mg IM every 28 days. The primary endpoint was clinical/radiographic PFS. At time of progression, patients were given the option to crossover to the alternate therapy. The trial was designed to show a 50% improvement in the primary endpoint for BAT vs. enzalutamide. The trial did not meet the primary endpoint with PFS of 5.62 m for the BAT arm and 5.72 m for the enzalutamide arm (p=0.2267), FIG. 7B. Objective response was 24.4% for BAT and 4.2% for enzalutamide (p=0.067). Best PSA50 was 26.4% for BAT and 25.5% for enzalutamide (p=0.697).

At time of progression, 46 (49%) of patients on the enzalutamide arm crossed over to receive BAT and 34 (33.7%) crossed over from BAT to receive enzalutamide and were available for analysis. Remarkably, the PSA50 response for patients who received enzalutamide after BAT was 72.7% compared to 25.5% for patients receiving enzalutamide immediately after abiraterone, FIG. 7D. Objective response in the enzalutamide post-BAT patients was 28.6% compared to 4.2% post-abiraterone, FIG. 7B. Time to PSA progression on enzalutamide was also markedly improved, increasing almost 3-fold from 3.8 months immediately post-abiraterone to 10.9 months post-BAT, FIG. 7B. Finally, post-hoc analysis of PSA progression to the first and second stages of the study (PSA PFS2) for all patients, including censored patients who did not crossover, revealed a median of 14.2 months for the sequence of enzalutamide crossing over to BAT, but 28.2 months for sequence of BAT crossing over to Enzalutamide, FIG. 7E.

### E. Sequential Hormone Therapy

Without wishing to be bound to any one particular theory, it is thought that a significant clinical response can be achieved in men with long standing castration resistant prostate cancer by rapidly cycling from the polar extremes of high dose to castrate serum levels of androgen. This approach is referred to as Bipolar Androgen Therapy (BAT). Beer et al., 2014. By pursuing this treatment strategy, high AR-expressing CRPC cells will be sensitive to killing by high dose levels of testosterone according to mechanisms described above. Those cells that try to adapt to high androgen by dropping AR expression to low levels will then become sensitive to killing when testosterone levels are lowered to near castrate levels.

In some embodiments, the presently disclosed subject matter provides method for treating prostate cancer in a subject in need of treatment thereof, the method comprising:
(a) administering to the subject a first dose of an androgen, or a derivative thereof, at a beginning of a first treatment cycle and a second dose, and optionally a third dose, of an androgen, or a derivative thereof, at a predetermined interval during the first treatment cycle, or if the third dose is administered, at a predetermined interval during a second treatment cycle; and
(b) sequentially administering one or more antiandrogens, one or more androgen synthesis inhibitors, or a combination thereof, at a completion of the first treatment cycle and throughout the second treatment cycle, and optionally throughout a third treatment cycle.

In certain embodiments, the method includes a cycling interval of about 1 to 3 months, including 1, 2, or 3 months. In such embodiments, androgen is administered one month then antiandrogen one month or the method could include 2 months or 3 months of each. In other embodiments, the administration switches from androgen to antiandrogen at the time of an increase in the subject's blood PSA level. In such embodiments, the length of treatment with both androgen or antiandrogen would be variable. Thus, the presently disclosed methods include 1, 2 or 3 months of androgen treatment followed by equal duration of 1, 2 or 3 months of antiandrogen.

In certain embodiments, the second dose, or optionally the third dose, of an androgen, or a derivative thereof, is administered about 28±5 days after the beginning of the first treatment cycle or, if the third dose is administered, about 28±5 days after the beginning of the second treatment cycle.

In more certain embodiments, the method further comprises starting administration of the one or more antiandrogens, one or more androgen synthesis inhibitors, or a combination thereof, at the end of a one-, two-, or three-month androgen treatment cycle.

In certain embodiments, the method comprises administering the one or more antiandrogens, one or more androgen synthesis inhibitors, or a combination thereof, each for one, two, or three 28±5 day treatment cycles.

In more certain embodiments, the method further comprises discontinuing administration of the one or more antiandrogens, one or more androgen synthesis inhibitors, or a combination thereof, at a completion of the second treatment cycle, or optionally at a completion of the third treatment cycle, and restarting the first treatment cycle comprising administering to the subject a first dose of an androgen, or a derivative thereof, at a beginning of a first treatment cycle and a second dose, and optionally a third dose, of an androgen, or a derivative thereof, at a predetermined interval during the first treatment cycle, or if the third dose is administered, at a predetermined interval during a second treatment cycle.

In certain embodiments, the method comprises alternating the first treatment cycle and the second treatment cycle, and optionally the third treatment cycle, until a clinical and/or radiographic progression is observed.

In other embodiments, the presently disclosed subject matter provides a method for treating prostate cancer in a subject in need of treatment thereof, the method comprising:
(a) administering to the subject a first dose of an androgen, or a derivative thereof, at a beginning of a first treatment cycle;
(b) measuring a prostate-specific antigen (PSA) level of the subject; and
(c) one of:
   (i) maintaining a subject exhibiting a declining PSA level or no PSA progression on the first treatment cycle until PSA progression is observed; or
   (ii) discontinuing the first treatment cycle in a subject exhibiting a PSA progression (≥25% increase in PSA from baseline) and starting a sequential treatment cycle comprising administering to the subject one or more antiandrogens, one or more androgen synthesis inhibitors, or a combination thereof.

In certain embodiments, the second or third dose of an androgen, or a derivative thereof, is administered about 28±5 days, including 23, 24, 25, 26, 27, 28, 29, 30, 31, 32, and 33 days, after the beginning of the first or second (i.e., previous) treatment cycle.

In certain embodiments, the method further comprises continuing administration of the one or more antiandrogens, one or more androgen synthesis inhibitors, or a combination thereof, until PSA progression is observed.

In particular embodiments, the method comprises administering the one or more antiandrogens, one or more androgen synthesis inhibitors, or a combination thereof, each day of the second treatment cycle for a total of about 56±5 days, including 51, 52, 53, 54, 55, 56, 57, 58, 59, 60, and 61 days.

In more certain embodiments, the method further comprises discontinuing administration of the one or more antiandrogens, one or more androgen synthesis inhibitors, or a combination thereof, once the subject exhibits PSA progression (≥25% increase in PSA from baseline) and restarting the first treatment cycle.

In yet more certain embodiments, the method further comprises alternating between the first treatment cycle and the second treatment cycle, and optionally the third treatment cycle, with onset of PSA progression until clinical and/or radiographic progression is observed.

In particular embodiments of the presently disclosed methods, the first dose of an androgen, or a derivative thereof, and the second dose of an androgen, or a derivative thereof, and optionally the third dose of an androgen, or a derivative thereof, are each sufficient to achieve a supraphysiological serum concentration of testosterone in the subject.

In certain embodiments, the supraphysiological serum concentration of testosterone in the subject is between about 3 to about 10 times a normal serum concentration of testosterone.

In particular embodiments, the serum concentration of testosterone is greater than about 1,500 ng/dL.

In certain embodiments of the presently disclosed methods, the androgen, or derivative thereof, is testosterone cypionate or testosterone enanthate at a dose of about 400 to about 500 mg.

In certain embodiments of the presently disclosed methods, one or more androgens having a different biological potency are administered to the subject, wherein the first dose of an androgen, or a derivative thereof, and the second dose of an androgen, or a derivative thereof, and optionally a third dose of an androgen, or a derivative thereof, are given at a dose range that achieves a same relative supraphysiologic potency as that achieved with testosterone cypionate or testosterone enanthate a dose of about 400 mg to about 500 mg.

In particular embodiments, the first dose of an androgen, or a derivative thereof, and the second dose of an androgen, or a derivative thereof, or optionally a third dose of an androgen, or a derivative thereof, each has a range from about 400 mg to about 500 mg, including 400, 405, 410, 415, 420, 425, 430, 435, 440, 445, 450, 455, 460, 465, 470, 475, 480, 485, 490, 495, and 500 mg.

In certain embodiments of the presently disclosed methods, the method comprises administering the androgen, or a derivative thereof, orally, transdermally or by intramuscular injection.

In particular embodiments, the androgen, or a derivative thereof, comprises an ester of testosterone or an ester of dihydrotestosterone.

In certain embodiments, the ester of testosterone of the ester of dihydrotestosterone is selected from a cypionate, enanthate, propionate, butyrate, and undecanoate ester of testosterone or dihydrotestosterone.

In particular embodiments, the androgen, or derivative thereof, is testosterone cypionate or testosterone enanthate.

One of ordinary skill in the art would recognize that other androgens, derivatives thereof, including prodrugs, could be used in the presently disclosed methods.

As used herein, the term “androgen” refers to any natural or synthetic steroid hormone that regulates the development and maintenance of male characteristics in vertebrates by binding to androgen receptors. The major androgen in males is testosterone. Other natural androgens include dehydroepiandrosterone (DHEA), which also is referred to as dehydroisoandrosterone or dehydroandrosterone, androstenedione (A4), androstenediol (A5), and dihydrotestosterone (DHT).

Anabolic steroids include natural androgens, such as testosterone, as well as synthetic androgens that are structurally related and have similar effects to testosterone. More particularly, anabolic steroids include testosterone and esters thereof, including, but not limited to, testosterone undecanoate, testosterone enanthate, testosterone cypionate, and testosterone propionate, dihydrotestosterone and esters thereof, including, but not limited to, dihydrotestosterone undecanoate, dihydrotestosterone enanthate, dihydrotestosterone cypionate, and dihydrotestosterone propionate; nandrolone esters, including nandrolone decanoate and nandrolone phenylpropionate; stanozolol; and metandienone (methandrostenolone). Other anabolic steroids include danazol, ethylestrenol, methyltestosterone, norethandrolone, oxandrolone, mesterolone, and oxymetholone, as well as drostanolone propionate (dromostanolone propionate), metenolone (methylandrostenolone) esters, including metenolone acetate and metenolone enanthate, fluoxymesterone, boldenone undecylenate, trenbolone acetate, and esters of DHT. Other anabolic steroids include 1-testosterone (dihydroboldenone), methasterone, trenbolone enanthate, desoxymethyltestosterone, tetrahydrogestrinone, and methylstenbolone.

In some embodiments, the androgen is an ester of testosterone or an ester of another anabolic steroid. Esters of testosterone include, but are not limited to, testosterone caproate, testosterone cypionate, testosterone decanoate, testosterone enanthate, testosterone isobutyrate, testosterone isocaproate, testosterone phenylpropionate, testosterone propionate, testosterone undecanoate, testosterone acetate, testosterone cyclohexylpropionate, testosterone enantate benzilic acid hydrazone, testosterone furoate, testosterone hexahydrobenzoate, testosterone hexahydrobenzylcarbonate, testosterone hexyloxyphenylpropionate, testosterone ketolaurate, testosterone nicotinate, testosterone phenylacetate, testosterone phosphate, testosterone undecylenate, testosterone valerate, testosterone buciclate, polytestosterone phloretin phosphate, testosterone 17β-(1-((5-(aminosulfonyl)-2-pyridinyl) carbonyl)-L-proline) (EC586), testosterone acetate butyrate, testosterone acetate propionate, testosterone benzoate, testosterone butyrate, testosterone diacetate, testosterone dipropionate, testosterone formate, testosterone isovalerate, testosterone palmitate, testosterone phenylbutyrate, testosterone stearate, testosterone sulfate, and dihydrotestosterone esters.

Esters of dihydrotestosterone (DHT; androstanolone, stanolone) include, but are not limited to, androstanolone benzoate, androstanolone enantate, androstanolone propionate, androstanolone valerate, dihydrotestosterone acetate, dihydrotestosterone butyrate, dihydrotestosterone formate, dihydrotestosterone undecanoate, and testifenon (chlorphenacyl DHT ester).

Esters of other natural anabolic steroids include, but are not limited to, androstenediol dipropionate, prasterone enantate, prasterone sulfate, androstenediol 3β-acetate, androstenediol 3β-acetate 17β-benzoate, androstenediol 17β-acetate, androstenediol diacetate, sturamustine,

Esters of synthetic AAS include methandriol esters, including methandriol bisenanthoyl acetate, methandriol dipropionate, methandriol propionate, and methandriol diacetate; nandrolone esters, including nandrolone decanoate, nandrolone phenylpropionate, nandrolone caproate, nandrolone cyclohexanecarboxylate, nandrolone cyclohexylpropionate, nandrolone cypionate, nandrolone furylpropionate, nandrolone hexyloxyphenylpropionate, nandrolone hydrogen succinate, nandrolone laurate, nandrolone propionate, nandrolone sulfate, nandrolone undecanoate, nandrolone 17β-adamantoate, nandrolone acetate, nandrolone benzoate, nandrolone cyclotate, nandrolone enanthate, nandrolone formate, and LS-1727; trenbolone ester, including trenbolone acetate, trenbolone hexahydrobenzylcarbonate, trenbolone enantate, trenbolone undecanoate

Esters of other synthetic AAS include bolandiol dipropionate, bolazine capronate, boldenone acetate, boldenone cypionate, boldenone propionate, boldenone undecylenate (boldenone undecenoate), clostebol acetate, clostebol caproate, clostebol propionate, drostanolone propionate, metenolone acetate, metenolone enantate, norclostebol acetate, oxabolone cypionate, propetandrol (norethandrolone 3β-propionate), stenbolone acetate, 11β-Methyl-19-nortestosterone dodecylcarbonate, dimethandrolone buciclate, dimethandrolone dodecylcarbonate, dimethandrolone undecanoate, mesterolone cypionate, nisterime acetate, trestolone acetate, and trestolone enantate.

In addition to esters, the presently disclosed methods can include ethers of androgens. Ethers of natural AAS include cloxotestosterone acetate, cloxotestosterone, and silandrone. Ethers of synthetic AAS include mepitiostane, methyltestosterone 3-hexyl ether, penmesterol, quinbolone, mesabolone, methoxydienone (methoxygonadiene), and prostanozol.

Androgens as can include derivatives of testosterone and other natural or synthetic androgens, including ester and ether prodrugs, and prohormones. Testosterone derivatives include 4-hydroxytestosterone, 11-ketotestosterone, Δ1-testosterone, and 4-Chlorotestosterone. Prohormone-like androgens include 4-androstenediol, 4-dehydroepiandrosterone (4-DHEA), 5-androstenedione, 5-dehydroandrosterone (5-DHA), 11β-hydroxyandrostenedione (11β-OHA4), 11-keto-4-androstenedione, 5-androstenediol, 4-androstenedione, 1-methyl-δ1-4-androstenedione, δ1-4-Androstenedione, dehydroepiandrosterone (DHEA, 5-DHEA), 6-methylidene-δ1-4-androstenedione, 4-hydroxy-4-androstenedione, 10-propargyl-4-androstenedione,

Prodrugs, including ethers, such as cloxotestosterone, quinbolone, and silandrone.

Dihydrotestosterone derivatives, including dihydrotestosterone (DHT), 4,5α-Dihydro-δ1-testosterone, 11-Ketodihydrotestosterone (11-KDHT), 2α-Methyl-4,5α-dihydrotestosterone, 2α,3α-Epithio-3-deketo-4,5α-dihydrotestosterone, 1α-Methyl-4,5α-dihydrotestosterone, 1-Methyl-4,5α-dihydro-δ1-testosterone, 2α-Chloro-4,5α-dihydrotestosterone 3-O-(p-nitrophenyl) oxime, and 2-Methyl-4,5α-dihydro-δ1-testosterone.

Prohormone-like dihydrotestosterone derivatives including 1-androsterone (1-Andro, 1-DHEA), 1-androstenediol (4,5α-dihydro-δ1-4-androstenediol), 1-androstenedione (4,5α-dihydro-δ1-4-androstenedione), 3-deketo-4,5α-dihydro-δ2-4-androstenedione), and epiandrosterone. Ether prodrugs including mepitiostane (2α,3α-Epithio-3-deketo-4,5α-dihydrotestosterone 17β-(1-methoxycyclopentane) ether), mesabolone (4,5α-Dihydro-δ1-testosterone 17β-(1-methoxycyclohexane) ether), and prostanozol (2′H-5α-Androst-2-eno[3,2-c]pyrazol-17β-ol 17β-tetrahydropyran ether). Azine dimers, including bolazine (3,3-[(1E,2E)-1,2-Hydrazinediylidene]di(2α-methyl-5α-androstan-17β-ol)).

19-Nortestosterone (nandrolone) derivatives including 19-Nortestosterone, 11β-Methyl-19-nortestosterone (11β-MNT), dienolone (19-Nor-89-testosterone), dimethandrolone (7α,11β-Dimethyl-19-nortestosterone), norclostebol (4-chloro-19-nortestosterone), oxabolone (4-hydroxy-19-nortestosterone, trenbolone (19-Nor-δ9,11-testosterone), and trestolone (MENT) (7α-Methyl-19-nortestosterone). Prohormone-like (nandrolone) derivatives including 7α-Methyl-19-nor-4-androstenedione (MENT dione, trestione), 19-Nor-5-androstenediol, 19-Nor-5-androstenedione, 19-Nordehydroepiandrosterone, bolandiol (nor-4-androstenediol), bolandione (nor-4-androstenedione), dienedione (nor-4,9-androstadienedione), methoxydienone (18-Methyl-19-nor-δ2,5 (10)-epiandrosterone 3-methyl ether), and trendione (nor-4,9,11-androstatrienedione). Ester prodrugs including olmantalate (19-Nortestosterone 17β-adamantoate).

17α-Alkylated testosterone derivatives including bolasterone (7α, 17α-dimethyltestosterone), calusterone (7β,17α-dimethyltestosterone), chlorodehydromethyltestosterone (CDMT) (4-chloro-17α-methyl-δ1-testosterone), enestebol (4-hydroxy-17α-methyl-δ1-testosterone), ethyltestosterone (17α-ethyltestosterone), fluoxymesterone (9α-fluoro-11β-hydroxy-17α-methyltestosterone), formebolone (2-formyl-11α-hydroxy-17α-methyl-δ1-testosterone), hydroxystenozole (17α-methyl-2′H-androsta-2,4-dieno[3,2-c]pyrazol-17β-ol), metandienone (17α-methyl-δ1-testosterone), methylclostebol (4-chloro-17α-methyltestosterone), methyltestosterone (17α-methyltestosterone), oxymesterone (4-Hydroxy-17α-methyltestosterone), and tiomesterone (1α,7α-Diacetylthio-17α-methyltestosterone). Prohormone-like 17α-Alkylated testosterone derivatives including chlorodehydromethylandrostenediol (CDMA) (4-Chloro-17α-methyl-δ1-4-androstenediol), chloromethylandrostenediol (CMA) (4-chloro-17α-methyl-4-androstenediol), methandriol (17α-methyl-5-androstenediol). Ether prodrugs, including methyltestosterone 3-hexyl ether (17α-methyl-4-hydro-δ3,5-testosterone 3-hexyl ether) and penmesterol (17α-Methyl-4-hydro-δ3,5-testosterone 3-cyclopentyl ether).

17α-Alkylated dihydrotestosterone derivatives including androisoxazole (17α-methyl-5α-androstano[3,2-c]isoxazol-17β-ol), desoxymethyltestosterone (3-deketo-17α-methyl-4,5α-dihydro-δ2-testosterone), furazabol (17α-methyl-5α-androstano[2,3-c][1,2,5]oxadiazol-17β-ol), mestanolone (methyl-DHT) (17α-methyl-4,5α-dihydrotestosterone), methasterone (2α,17α-Dimethyl-4,5α-dihydrotestosterone), methyl-1-testosterone (17α-methyl-4,5α-dihydro-δ1-testosterone), methyldiazinol (3-azi-17α-methyl-4,5α-dihydrotestosterone), methylepitiostanol (2α,3α-epithio-3-deketo-17α-methyl-4,5α-dihydrotestosterone), methylstenbolone (2,17α-dimethyl-4,5α-dihydro-δ1-testosterone), oxandrolone (2-oxa-17α-methyl-4,5α-dihydrotestosterone), oxymetholone (2-hydroxymethylene-4,5α-dihydro-17α-methyltestosterone), and stanozolol (17α-methyl-2′H-5α-androst-2-eno[3,2-c]pyrazol-17β-ol. Azine dimers including mebolazine (3,3-[(1E,2E)-1,2-Hydrazinediylidene]di(2α,17α-dimethyl-5α-androstan-17β-ol).

17α-Alkylated 19-nortestosterone derivatives including dimethyltrienolone (7α,17α-Dimethyl-19-nor-δ9,11-testosterone), dimethyldienolone (7α,17α-Dimethyl-19-nor-89-testosterone, ethyldienolone (17α-ethyl-19-nor-89-testosterone), ethylestrenol (17α-Ethyl-3-deketo-19-nortestosterone), methyldienolone (17α-Methyl-19-nor-89-testosterone), methylhydroxynandrolone (MOHN, MHN) (4-Hydroxy-17α-methyl-19-nortestosterone), metribolone (methyltrienolone, R-1881) (17α-methyl-19-nor-δ9,11-testosterone), mibolerone (7α,17α-dimethyl-19-nortestosterone), norboletone (17α-ethyl-18-methyl-19-nortestosterone), norethandrolone (17α-ethyl-19-nortestosterone), normethandrone (17α-methyl-19-nortestosterone), RU-2309 (17α,18-dimethyl-19-nor-δ9,11-testosterone), and tetrahydrogestrinone (THG) (17α-ethyl-18-methyl-19-nor-δ9,11-testosterone). Prohormone-like 17α-alkylated 19-nortestosterone derivatives including bolenol (ethylnorandrostenol) (3-Deketo-17α-ethyl-19-nor-5-androstenediol). Ester prodrugs including propetandrol (17α-Ethyl-19-nortestosterone 3-propionate).

17α-Vinylated testosterone derivatives including vinyltestosterone (17α-ethenyltestosterone). 17α-vinylated 19-nortestosterone derivatives including 17α-ethenyl-19-nortestosterone.

17α-Ethynylated testosterone derivatives including ethisterone (17α-ethynyltestosterone), danazol (2,3-isoxazol-17α-ethynyltestosterone).

17α-Ethynylated 19-nortestosterone derivatives including norethisterone (17α-ethynyl-19-nortestosterone), etynodiol (17α-ethynyl-3-deketo-3β-hydroxy-19-nortestosterone), gestrinone (ethylnorgestrienone, R-2323) (17α-Ethynyl-18-methyl-19-nor-89,11-testosterone), levonorgestrel ((−)-norgestrel) ((−)-17α-Ethynyl-18-methyl-19-nortestosterone), lynestrenol (17α-ethynyl-3-deketo-19-nortestosterone), norgestrel (17α-ethynyl-18-methyl-19-nortestosterone, norgestrienone (17α-ethynyl-19-nor-δ9,11-testosterone), and tibolone (7α-methyl-17α-ethynyl-19-nor-δ5 (10)-testosterone. Ethers including quingestanol (4-hydro-19-nor-δ3,5-testosterone 3-cyclopentyl ether). Esters including etynodiol diacetate (17α-ethynyl-3-deketo-3β-hydroxy-19-nortestosterone 3β,17β-diacetate), norethisterone acetate (17α-ethynyl-19-nortestosterone 17β-acetate), and norethisterone enanthate (17α-ethynyl-19-nortestosterone 17β-enanthate). Ethers and esters including quingestanol acetate (4-hydro-17α-ethynyl-19-nor-δ3,5-testosterone 3-cyclopentyl ether 17β-acetate).

In certain embodiments of the presently disclosed methods, the one or more antiandrogens is selected from the group consisting of bicalutamide, flutamide, nilutamide, apalutamide, darolutamide, enzalutamide, cyproterone acetate, proxalutamide, cimetidine, and topilutamide.

In particular embodiments, the one or more antiandrogens is selected from the group consisting of enzalutamide, apalutamide, darolutamide, and combinations thereof.

In yet more particular embodiments, the one or more antiandrogens is enzalutamide.

In certain embodiments of the presently disclosed methods, the one or more androgen synthesis inhibitors is selected from the group consisting of a CYP17A1 inhibitor, a CYP11A1 (P450scc) inhibitor, a 5α-Reductase inhibitor, and combinations thereof.

In particular embodiments, the one or more androgen synthesis inhibitors is selected from the group consisting of abiraterone acetate, ketoconazole, seviteronel, aminoglutethimide, alfatradiol, dutasteride, epristeride, finasteride, and combinations thereof.

In more particular embodiments, the one or more androgen synthesis inhibitors is abiraterone acetate.

In certain embodiments of the presently disclosed methods, the one or more antiandrogens, one or more androgen synthesis inhibitors, or a combination thereof, are administered at a dosage having a range selected from the group between about 100 to about 200 mg/day, between about 110 to about 190 mg/day, between about 120 to about 180 mg/day, between about 130 to about 170 mg/day, and about 160 mg per day.

In other embodiments, the presently disclosed method further comprises concurrently administering an androgen deprivation therapy (ADT) to the subject. In certain embodiments, the ADT comprises surgical castration or administering a luteinizing hormone-releasing hormone (LHRH) agonist or a LHRH antagonist to the subject. In more certain embodiments, the LHRH agonist is selected from the group consisting of leuprolide (Lupron, Eligard), goserelin (Zoladex), triptorelin (Trelstar), and histrelin (Vantas). In more certain embodiments, the LHRH antagonist is selected from the group consisting of Degarelix (Firmagon) and Relugolix (Orgovyx).

In certain embodiments of the presently disclosed methods, the method further comprises administering immune checkpoint blockade therapy to the subject if the subject exhibits clinical and/or radiographic progression.

In particular embodiments, the immune checkpoint blockade therapy comprises administering an anti-PD1/PDL1 antibody or an anti-CTLA4 antibody.

In more particular embodiments, the anti-PD1/PDL1 antibody is selected from the group consisting of pembrolizumab, nivolumab, and atezolizumab.

In more particular embodiments, the anti-CTLA4 antibody comprises ipilimumab.

In certain embodiments of the presently disclosed methods, the subject has progressive prostate cancer after treatment with abiraterone in combination with androgen deprivation therapy (ADT) as an initial therapy or as a second-line therapy after development of resistance to primary ADT.

In certain embodiments of the presently disclosed methods, the prostate cancer comprises castration resistant metastatic prostate cancer.

In certain embodiments of the presently disclosed methods, the subject is asymptomatic.

In other embodiments of the presently disclosed methods, the subject is symptomatic.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Sequential High Dose Androgen and Antiandrogen as Sequential Therapy to Improve Survival and Enhance Response to Immune Therapy for Castrate Resistant Prostate Cancer

The presently disclosed subject matter provides a method for treating prostate cancer using sequential and repeated therapy with high dose testosterone and antiandrogen as a means to enhance and prolong hormonal response to improve survival of men with prostate cancer as well as enhance response to immune therapy in men with prostate cancer.

Inhibition of androgen receptor (AR) function through androgen deprivation (ADT) and direct AR inhibition has been the mainstay of treatment for advanced prostate cancer since its discovery by Huggins 80 years ago. Denmeade and Isaacs, 2010. ADT remains the most effective therapy for any human cancer, with response rates upwards of 95% and response duration that can last for decades in some men. ADT, however, is not curative and all men eventually progress to a state known as castrate-resistant prostate cancer (CRPC). Isaacs et al., 2012. At this stage, men are treated with a next-generation second-line hormone therapy [i.e., CYP17 inhibitor abiraterone (Abi), or antiandrogen enzalutamide (Enza)], which produces response in about 60-70% of patients.

Median survival after progression on a second hormone therapy is approximately 18-20 months. Hormonal resistance increases with each subsequent line of therapy, as only approximately 20% of patients respond to third-line treatment. Thus, standard treatment for patients progressing on second-line hormone therapy has not been defined. Patients can be offered docetaxel chemotherapy or ²²³Radium, but response duration is short with modest (i.e., few months) effect on survival.

Further, prostate cancer is considered an immunologically “cold” disease, and thus, immune checkpoint inhibition has not been effective. One goal of the presently disclosed subject matter is to establish high-dose testosterone (T) in sequence with Enza (BEST) as the standard treatment option for metastatic CRPC progressing on second-line hormonal therapy. A second goal is to demonstrate that BEST can activate the immune microenvironment to induce immunotherapy responsiveness.

Preclinical studies have demonstrated that the growth of human CRPC cells can be inhibited by exposure to supraphysiologic levels of testosterone. Importantly, these studies also demonstrate that PCa cells can develop therapeutic resistance due to their ability to adaptively upregulate AR levels in response to low-androgen growth conditions. It has been demonstrated that the marked upregulation of AR produces a therapeutic vulnerability to high androgen levels. Based on these preclinical data, a clinical program to test these concepts was instituted. Four clinical studies in approximately 250 men have been completed in which the safety and efficacy of rapid cycling between the polar extremes of supraphysiologic and near-castrate serum testosterone (T) levels, a concept we have termed “Bipolar Androgen Therapy” (BAT), has been demonstrated in asymptomatic men with metastatic CRPC. The overall key findings from these clinical studies have been that BAT (a) could be safely administered; (b) did not produce symptomatic disease progression; (c) produced sustained PSA and objective responses, and (d) re-sensitized fand induced responses to subsequent antiandrogen therapy.

As an example of this potential for overcoming resistance and improving survival, in the TRANSFORMER study, 195 men with CRPC progressing on second-line Abi were randomized to receive either third-line Enza or BAT with crossover at time of progression. The PSA50 response in those receiving third-line Enza was 25% and the time to PSA progression was 3.8 months. In contrast, in men who crossed over to Enza after first receiving BAT, the PSA50 response to Enza was 78% and the time to PSA progression was 11 months. The overall survival for men who received enzalutamide alone was 28 months while for men who first received BAT and then enzalutamide survival was 37 months.

Further, immune checkpoint blockade has become an important part of the treatment paradigm for many cancer types. Unfortunately, results to date have been disappointing in patients with metastatic CRPC with response rates <15% across multiple small studies. In the KEYNOTE-199 objective response to Pembrolizumab was <5% in 258 patients with mCPRC treated previously with docetaxel and one or more hormonal therapies. Interestingly, recent data suggest that high-dose T increases density of tumor-infiltrating lymphocytes (TILs).

As part of a longitudinal follow-up of patients on the RESTORE and TRANSFORMER studies, three patients were identified who had dramatic response to immunotherapy. Across the two studies, only these three patients had these characteristics. None of these patients had MSI or mismatch repair gene mutations. These results led us to explore the effects of BAT on activating an immune response in PCa models. In these studies, it was demonstrated that high-dose testosterone can activate the cGAS-STING pathway as well as the NFκB pathway inducing downstream interferon-stimulated genes (3C) and inflammatory cytokines such as CXCL10, which can attract both NK cells and CD8+ into the tumor microenvironment.

While ADT for advanced PCa often produces debilitating sexual and metabolic side effects, a significant feature of this approach is that BAT can make men feel remarkably better by decreasing fatigue, increasing physical activity and restoring libido and sexual function. High-dose T also produced favorable effects on body composition by increasing skeletal muscle mass and decreasing subcutaneous and visceral fat. Thus, incorporation of high-dose T into the treatment paradigm has the potential to improve the quality of life of PCa patients and minimize the morbidity from the metabolic sequelae produced by ADT.

Without wishing to be bound to any one particular theory, it is thought that sequential therapy with supraphysiologic testosterone and antiandrogen, such as enzalutamide is a new treatment strategy to disrupt adaptive AR autoregulation, thereby enhancing and prolonging response and mitigating side effects associated with ADT. Based on preclinical and clinical data, a second hypothesis is that sequential therapy will produce an activated immune microenvironment resulting in enhanced response to immune checkpoint blockade, which thus far has been ineffective in prostate cancer.

### Example 2

Treatment Protocol for Sequential High Dose Testosterone and Enzalutamide in Asymptomatic Men with Castration Resistant Metastatic Prostate Cancer

Asymptomatic men progressing metastatic CRPC after treatment with ADT+abiraterone combination or sequential ADT followed by abiraterone will be treated to determine if alternating treatment with intramuscular testosterone given on a dose/schedule designed to result in rapid cycling from the polar extremes of high dose to near castrate levels (i.e., Bipolar Androgen Therapy (BAT)) and enzalutamide every 2 months (STE) or at time of PSA progression (VSTE) will improve primary and secondary objectives vs. continuous enzalutamide as standard therapy.

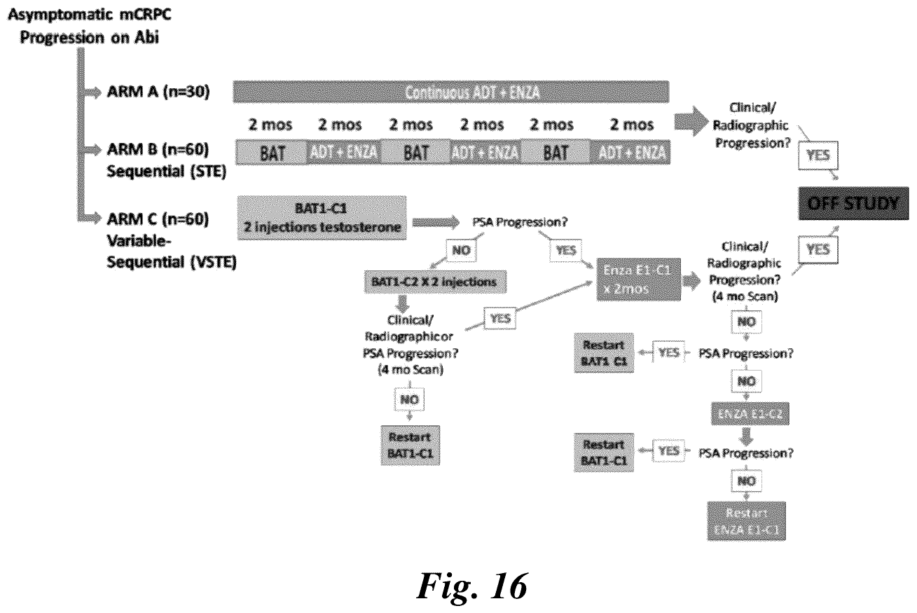

A schematic providing the presently disclosed dosage regimen is shown in FIG. 16.

In the presently disclosed dosage regimen, the subject will continue on ADT with a LHRH agonist including, but not limited to Zoladex, Trelstar, Eligard or Lupron, or a LHRH antagonist (e.g., Degarelix) if not surgically castrated throughout the duration of the treatment protocol to inhibit endogenous testosterone production.

In one embodiment, the subject will receive continuous therapy with standard dose enzalutamide (e.g., 160 mg po q day).

In another embodiment, the subject will receive Sequential Testosterone and Enzalutamide (STE). In this embodiment, the subject will receive intramuscular injection with testosterone cypionate (T) at an FDA-approved dose of 400 mg every 28 days×2 (i.e., cycle 1). This dose was selected based on data demonstrating that it produces an initial high dose serum level of testosterone (i.e., >1500 ng/dL or 3-10 times normal level) with eugonadal levels achieved at the end of two weeks and near castrate levels after 28 days. On Day 1 of cycle 2, patients will stop testosterone and begin enzalutamide 160 mg po q day for 56 days. Each cycle is 56 days. On Day 1 of cycle 3, the subject will not take enzalutamide and will again receive injection of testosterone. The subject will continue to alternate one cycle of testosterone (2 injections) with one cycle of 56 days of enzalutamide.

In yet another embodiment, the subject will receive Variable Sequential Testosterone and Enzalutamide (VSTE). In this embodiment, the subject will receive intramuscular injection with testosterone cypionate (T) at an FDA-approved dose of 400 mg every 28 days×2 injections per cycle. The subject will remain on high dose testosterone for at least one cycle. Each cycle is 56 days. Subjects with declining PSA will remain on high dose testosterone for additional cycles of 2 injections until PSA progression occurs based on PCWG3 criteria. Subjects with PSA progression (≥25% increase in PSA from baseline) will stop testosterone injection. These subjects will then be started on Enzalutamide. Subjects on enzalutamide with PSA decline after one 56-day cycle will continue on Enzalutamide until PSA progression occurs. Subjects with PSA progression (≥25% increase in PSA from baseline) will stop Enzalutamide and will restart injections of testosterone with 2 injections/cycle. These cycles of switching between testosterone and enzalutamide with onset of PSA progression will continue until clinical and/or radiographic progression occurs.

In each embodiment, treatment will be given on indicated timepoints±5 days.

More particularly, the presently disclosed treatment protocol can include the following:

1. Subjects receiving enzalutamide only (Arm A) will have a clinic visits every cycle (8 weeks) to undergo assessment of toxicity, ECOG performance status and vital signs (temperature, blood pressure and heart rate);
2. Subjects receiving Sequential high dose testosterone and enzalutamide (Arm B) will be treated according to the following protocol:
a. Patients will begin testosterone cypionate injection on Day 1. Patients will receive testosterone cypionate every 28 days×2 (56 days/cycle);
b. Patients will stop testosterone and begin enzalutamide (Day 1 cycle 2). Patients will continue enzalutamide for 56 days/cycle;
c. Patient will undergo assessment of toxicity, ECOG performance status and vital signs (temperature, blood pressure and heart rate) each cycle;
   3. Subjects receiving Variable Sequential high dose testosterone and enzalutamide (VSTE) (Arm C) will be treated according to the following schedule:
a. Patients will begin testosterone cypionate injection on Day 1 (BAT1-C1). Patients will receive testosterone cypionate every 28 days×2;
b. After 1st cycle (BAT1-C1) patients are assessed for PSA progression:
   i. If no PSA progression patients continue for additional cycle of 2 injections of testosterone (BAT1-C2);
   ii. If PSA progression is observed after BAT1-C1, patients begin enzalutamide (E1-C1);
c. Patients proceeding to BAT1-C2 will have Bone and CT scan and evaluation after BAT1-C2:
   i. Patients with radiographic or PSA progression proceed to E1-C1;
   ii. Patients without radiographic or PSA progression recycle back to BAT1-C1;
d. Patients proceeding to E1-C1 will stop testosterone and begin enzalutamide. Patients will continue enzalutamide for 56 days/cycle;
e. After 1st cycle (E1-C1) patients are assessed for radiographic and PSA progression:
   i. Patients with radiographic progression will discontinue treatment protocol;
   ii. Patients without radiographic progression or PSA progression proceed to E1-C2;
   iii. Patients with PSA progression recycle back to BAT1-C1;
f. Patients proceeding to E1-C2 will complete a 56-day cycle of enzalutamide and then assessed for PSA progression;
   i. If PSA progression, patients recycle back to BAT1-C1;
   ii. If no PSA progression, patients recycle back to E1-C1;
      4. CBC and Comp Panel and PSA before each cycle for all arms. PSA may be performed at outside laboratory for patients as long as patient can have study done at the same outside lab each time.
      5. Arm B only:
a. Testosterone at C1 biopsy visit, C2V4, C4V7 and then each even cycle after C5;
b. An additional CBC and Comp panel collection after patient's first injection of testosterone in C1;
   6. Arm C only:
a. Testosterone at BAT1-C1 biopsy visit, pre BAT1-C2, post BAT1-C2 assessment, pre E1-C2 assessment and post E2-C2 assessment;
   7. CT scan, bone scan every 2 cycles (16 weeks).
   8. Blood drawn for research testing to assess level of full length and variant androgen receptor at progression/end of study visit for both Arm A and B, and at V4 and V5 for Arm B, and at pre BAT1-C2 assessment and pre E1-C2 assessment.
   9. Blood drawn for chemokines at V3 and at progression for Arm A and C1 biopsy visit, V4, V5, V7, V8 and at progression for Arm B, and at BAT1-C1 biopsy visit, post BAT1-C2 assessment, pre E1-C2 assessment and post E2-C2 assessment for Arm C.
   10. Blood drawn to evaluate change in cell populations in response to Testosterone and enzalutamide at V3 and at progression for Arm A and at C1 biopsy visit, V4, V5, V7, V8 and at progression for Arm B, and at BAT1-C1 biopsy visit, post BAT1-C2 assessment, pre E1-C2 assessment and post E2-C2 assessment for Arm C.
   11. Patients in Biopsy group B on Arm B and C will have testosterone level, plasma for chemokines and blood for PBMCs drawn on the Biopsy day
   12. Rectal swab collected at V4 for Arm A and V4 and V5 for Arm B and at pre BAT1-C2 assessment and pre E1-C2 assessment for Arm C.
   13. Quality of life surveys at C3 and End of Study for all Arms.

### Example 3

### Supraphysiological Testosterone Induces Ferroptosis and Activates NF-kB Mediated Immune Pathways in Prostate Cancer Through Nucleophagy

### 3.1 Overview

The discovery that androgens play an important role in the progression of prostate cancer has led to the development of androgen deprivation therapy (ADT) as a first line of treatment against PCa. However, paradoxical growth inhibition has been observed, both experimentally and clinically, in a subset of PCa upon administration of supraphysiological levels of testosterone. Here we report that SupraT activates cytoplasmic nucleic acid sensors and induces growth inhibition of SupraT-sensitive PCa cells. This is initiated by the induction of two parallel autophagy-mediated processes, namely, ferritinophagy and nucleophagy. Consequently, autophagosomal DNA activates nucleic acid sensors that converge on NF-kappaB to drive immune signaling pathways. Chemokines and cytokines secreted by the tumor cells in response to SupraT result in increased migration of cytotoxic immune cells to tumor beds of animal xenografts and patient tumors. Collectively, our findings indicate that SupraT may inhibit a subset of PCa by activating nucleic acid sensors and downstream immune signaling.

The presently disclosed subject matter demonstrates that supraphysiological testosterone induces two parallel autophagy-mediated processes, namely, ferritinophagy and nucleophagy. Consequently, autophagosomal DNA activates nucleic acid sensors to drive immune signaling pathways in prostate cancer.

### 3.2 Background

In 1941, Charles Huggins discovered the benefits of androgen deprivation therapy, which has become the mainstay for advanced prostate cancer (PCa) treatment. Huggins and Hodges, 1941. From the outset, however, it was recognized that all men eventually develop castration-resistant prostate cancer (CRPC). Harris et al., 2009. Evaluation of clinical specimens demonstrates that CRPC cells remain highly reliant on Androgen receptor (AR) signaling. Linja et al., 2001; Taplin and Balk, 2004. These studies suggested that the adaptive reliance on AR signaling by CRPC cells becomes a therapeutic liability that can be exploited through the administration of SupraT, a concept we have termed bipolar androgen therapy (BAT). Isaacs et al., 2012; Schweizer et al., 2015. In this regard, we and others have demonstrated that the growth of some AR-positive human PCa cells can be inhibited by exposure to SupraT. Schweizer et al., 2015. The mechanisms underlying this paradoxical effect of SupraT on PCa cells are likely multifactorial as the androgen is the key mediator of prostate cancer cell metabolism, proliferation and death. Haffner et al. and others showed that androgens generate double-strand DNA breaks (DSBs) in PCa cells through the recruitment of AR and topoisomerase II beta to androgen response elements. Haffner et al., 2010. Hypothetically, in prostate tumors with DNA repair mutations, SupraT-induced DSBs would trigger DNA repair stress and lead to either growth inhibition or cell death. Chatterjee et al., 2019. Intuitively, these tumors would be acutely susceptible to BAT. In agreement with this postulate, we have recently discovered an association between germline and/or somatic DNA repair gene mutations and favorable response to BAT. Teply and Antonarakis, 2017; Teply et al., 2017; Mark et al., 2021. A link between DNA repair gene mutation or transcriptome repression and response to BAT has also been reported by others. Chatterjee et al., 2019; Lam et al., 2020.

The presently disclosed subject matter demonstrates that, in some embodiments, SupraT induces ferroptosis and nucleophagy-mediated immune activation resulting in growth inhibition of PCa.

### 3.3 Material & Methods

### 3.3.1 Cell Culture

LNCaP, LAPC4, HEK293T, and NK92 cells were purchased from the American Type Culture Collection (ATCC). LNCaP, LAPC4, and 22Rv1 cells were cultured in phenol red-free RPMI (Thermo Fisher), and HEK293T cells were cultured in DMEM-high glucose (Sigma) supplemented with 10% FBS (Gemini Bio). VCaP cells were cultured in DMEM media (ATCC) containing 1.5 gram/L sodium-bi-carbonate. NK92 cells were cultured in CTS™ AIM VR SFM (Thermo Fisher) with 200 U/mL recombinant IL-2 (Peprotech), 12.5% horse serum (Thermo Fisher), and 12.5% FBS according to manufacturer's instructions. All cell lines were tested for mycoplasma using the PCR based mycoplasma detection kit (Agilent).

### 3.3.2 Knockout/Knockdown Cell Line Generation

To generate the gene knockouts, tetracycline induced cas9 vector (Addgene #50661) was stably expressed in prostate cancer lines. sgRNAs against target genes were cloned in pLXsgRNA vector (Addgene #50662), and lentiviral particles for sgRNA were produced in HEK293T cells by co-transfecting pLXsgRNA plasmid with pMD2.G (Addgene #12259) and psPAX2 (Addgene #12260). Viruses were harvested after 48 h, and cells were infected with lentiviral particles. Transduced cells were treated with 1 μg/mL doxycycline to induce cas9 before selection with 10 μg/mL blasticidin. Following selection, cells were transferred in 96-well plates to select individual clones. Knockouts were verified by western blots, and confirmed clones were expanded and cryopreserved for future experiments.

For NCOA4 knockdown in LNCaP cells, MISSION shRNA constructs (TRCN0000236184, TRCN0000236186, TRCN0000236187, TRCN0000236188 and TRCN000019724) were purchased from Sigma, and lentiviral particles were generated in HEK293T cells by co-transfecting shRNA plasmid construct with pMD2.G (Addgene #12259) and psPAX2 (Addgene #12260). LNCaP cells were infected with NCOA4 lentivirus and were selected with 1.0 μg/mL puromycin. The level of NCOA4 knockdown was measured by western blots, and confirmed cells were expanded, cryopreserved, and used for the experiments.

### 3.3.3 Quantitative Real-Time PCR

Total RNA was extracted from cells using Trizol reagent (Thermo Fisher) as per manufacturer's instructions. cDNA was synthesized from RNA by reverse transcription using SuperScript IV reverse (Thermo Fisher). Quantitative PCR was performed on samples mixed with SYBR master mix (BioRad) and gene-specific primers using a real-time thermocycler (BioRad). Data analysis was performed using the ΔΔCt method, and fold change (2\hat{ }⁻ΔΔ^{Ct}) was calculated after double normalization with the housekeeper gene and respective untreated control. Each PCR reaction was run in triplicates.

### 3.3.4 Immunoprecipitation

NCOA4-FLAG-HA (CTAP) plasmid was received as a gift from Prof. J Wade Harper (Harvard Medical School, Boston). LNCaP cells were transfected with NCOA4-FLAG plasmid for immunoprecipitation studies or kept as untransfected control (C). NCOA4-FLAG transfected cells were either treated with 10 nM R1881 (T) or kept as untreated control (C*). Following treatment, cells were washed with 3 mL PBS, harvested in ice-cold PBS, and pelleted using a refrigerated centrifuge. Cells were lysed with 750 μL 1× lysis buffer (Promega) containing protease and phosphatase inhibitor for 20 minutes on ice. After lysis, the lysates were centrifuged at 13200×g at 4° C., and 10 percent of supernatant was stored as input. The remaining supernatant was used for immunoprecipitation using anti-FLAG M2 agarose beads (Thermo Fisher) as per manufacturer's instructions. Affinity separated FLAG-tagged protein was eluted using 35 μL IgG elution buffer (Thermo Fisher), denatured with an equal volume of Laemmli buffer, and stored at −80° C. before western blot analysis. Each experiment was repeated at least three times.

### 3.3.5 Western Blotting

Cell lysates were prepared either in 1× denaturing lysis buffer (Cell Signaling #9803) or non-denaturing lysis buffer (50 mM HEPES pH 7.4, 150 mM NaCl, 5 mM EDTA, 10% glycerol, 1% Nonidet P-40, and supplemented with phosphatase and protease inhibitors). For denaturing gels, lysates were mixed with Laemmli buffer containing β-mercaptoethanol, boiled for 5 mins and stored at −80° C. until use. Native lysates were mixed with native loading buffer (BioRad), and semi-native lysates were mixed with Laemmli buffer without beta-mercaptoethanol and stored at −80° C. without boiling. Native gels were resolved as described previously, Robitaille et al., 2016; denaturing and semi-native gels were resolved on 4-15% precast polyacrylamide gels (BioRad) followed by transfer to PVDF membrane. Membranes were blocked in 5% nonfat milk in wash buffer (Tris-buffered saline+0.1% Tween-20). After blocking, immunoblotting was performed with primary antibodies overnight at 4° C., followed by incubation with HRP-conjugated secondary antibodies for 1 h at room temperature. Blots were analyzed using the chemiluminescence method. Blots were developed using ECL Western blot detection reagent (GE Healthcare) for highly expressed proteins and Super Signal West Femto (Thermo Fisher) reagent for low expression proteins. Each experiment was repeated at least two to three times.

### 3.3.6 Immunofluorescence

Cells were grown on sterile coverslips in low density and were treated with R1881 (T) or vehicle control (C). After treatment, media was removed, and cells were fixed and permeabilized using chilled methanol for 10 min in deep-freezer followed by 10 min fixation in 10% natural buffered formalin at room temperature. Post-fixation, cells were blocked overnight in sterile-filtered 5% BSA in PBS at 4° C. Cells were incubated with a primary antibody, followed by a suitable fluorochrome tagged secondary antibody at 4° C. overnight. Both primary and secondary antibodies were diluted with 5% BSA in PBS. Cells were postfixed with 10% NBF for 2-3 min before mounting on a clean glass slide using Vectashield antifade mounting media (Vector Laboratories). Image acquisition was performed on the LSM 700 laser confocal microscope (Zeiss). Experiment was repeated at least three times and images from at least five random fields from each experiment were used for analysis. Images were analyzed using the image processing package Fiji.

### 3.3.7 Measurement of Cell Death

Cells were stained with 0.4% trypan blue (Sigma) and incubated for 2-3 min at room temperature to allow dead cells to take up the blue stain. Cell viability was measured using an automated cell counter, Cellometer, (Nexcelom). Each experiment was repeated at least three times. Dead cells were calculated from subtracting the percentage of viable cells from 100. Mean percentage of dead cells was plotted on histograms.

### 3.3.8 Measurement of the Labile Iron Pool (Fe²⁺)

An iron assay kit (Sigma) was used to measure the labile iron pool in control and R1881 treated cells. LNCaP and LAPC4 cells were culture in a 100-mm tissue culture dish and were treated for 72 before lysis in the buffer provided with the kit by snap freezing and thawing method. The lysate was cleared by centrifuging at 13000×g, and iron was measured by iron detection reagents supplied in the kit. Iron levels were calculated and normalized by the total protein present in the cell lysate. Levels of Fe²⁺ were analyzed in SupraT treated cells and were normalized to vehicle control cells.

### 3.3.9 NK Cell Migration Assay

This assay was performed in a trans-well co-culture set up, using tumor cells in the bottom chamber (culture plate) and NK-92 cells in the upper chamber (insert) (Corning). LNCaP and LAPC4 cells were first seeded in a 24-well plate for 24 h (day 1). The following day, tumor cells were treated with vehicle (C) or 10 nM R1881 (T) for 72 h. After 72 h, NK-92 cells, stained with 5 μM CFSE (Biolegend) for 10 min at 37° C., were then added to a 5-micron pore size insert placed in each culture well already containing the vehicle/R1881 treated tumor cells. The culture plate was incubated for 6 h, and microscopy was performed to image the CFSE labeled NK-92 cells that migrated to the lower chamber of the plate. Images were analyzed using the Image J software to determine the cell count. At least five fields were counted per experiment.

### 3.3.10 Cell Cycle Analysis

Harvested cell pellets were resuspended in ice-cold 70% ethanol and incubated at −20° C. overnight. Cells were then washed with 1×PBS, resuspended in 50 μg/mL propidium iodide (Sigma) in PBS with 50 μg/mL RNase A (Sigma), and incubated for 30 min at room temperature. Cell cycle analysis was performed using BD FACSCelesta cytometer equipped with BD FACS DIVA. Data were analyzed using the FlowJo software version 10 (FlowJo). The distribution of cells in the cell cycle phases was calculated using the Watson-Pragmatic algorithm. Each experiment was repeated at least three times.

### 3.3.11 NanoString Immune Profiling Assay

Total RNA was isolated from vehicle/R1881 treated cells using Trizol (Thermo Fisher), and RNA quality was determined using the Bioanalyzer (Agilent). Gene signature was determined by using the NanoString PanCancer Immune Profiling Panel according to the manufacturer's instructions (NanoString Technologies). House-keeping genes and built-in positive controls were used for data normalization and quality control assessments. Data analysis and gene expression analysis was performed using the nSolver 4.0 software (NanoString Technologies).

### 3.3.12 Luciferase Reporter Assay

Dual-Luciferase® reporter assay (Promega) was used to measure NF-kB, STING, and AIM-2 promoter activity. LNCaP and LAPC4 cells were transfected with Ig-IFN-Luc and Renila-Luc plasmids in 12-well plates and were incubated with vehicle or 10 nM R1881 treatment for 72 h. Post-treatment, cells were washed with PBS, followed by lysis with 250 μL 1× Passive lysis buffer (Promega) at room temperature for 15 min. The lysate was cleared by isolating cell debris by centrifugation and stored at −80° C. unless used immediately for luminescence measurement. For luminometric measurement, 100 μL LARII buffer containing D-luciferin was dispensed in each well of the luminometer plate followed by 20 μL of cell lysate to measure Firefly luminescence. After measuring firefly luminescence, 100 μL Stop & Glo® reagent containing quencher and substrate for Renilla luciferase was added to measure respective responsive luminescence. Data were normalized using individual well's Renilla luminescence. Each experiment was performed in triplicates and repeated three times.

### 3.3.13 Bioplex Assay for Chemokine and Cytokine Measurement

The concentration of 17 chemokines, cytokines, and growth factors in culture supernatants of control or R1881 treated LNCaP cells was determined using the Bioplex Pro Human Cytokine 17-plex assay system (Bio-Rad) according to manufacturer's protocol. Culture supernatant from vehicle and R1881 treated LNCaP, and LAPC4 cells were collected at 3 and 6-day time points, and stored at −80° C. until further analysis. Data was collected on the Bio-Rad BioPlex 200 instrument and analyzed using the Bio-Plex Manager (Bio-Rad Laboratory).

### 3.3.14 Autophagosome Flux Measurement

Cells were plated on 60-mm dishes to obtain 80% confluence on the next day. Cells were transfected with tandem mRFP-GFP fluorescent-tagged LC3 (ptfLC3) (Addgene #21074). 24 h post-transfection, cells were trypsinized and plated on sterile glass-bottom dishes at a 40-50% confluency and treated with androgens. Autophagosomes are labelled as green and red dual positive punctas and autolysosomes appear as red puncta as the green puncta is pH sensitive and quenched by the acidic lysosomal pH. Cells were then imaged using a live cell Zeiss LSM780-FCS Single-point, laser scanning confocal microscope. Images were processed using the ImageJ software. Cells with predominantly yellow RFP/GFP (autophagosome) or red RFP (autolysosome) punctae were counted and analyzed from at least twenty fields. Each experiment had images from at least five random fields and repeated three times.

### 3.3.15 Mouse Xenografts and Treatment

Using a Johns Hopkins Animal Care and Use Committee approved protocol, adult athymic nude mice were inoculated subcutaneously in the flank with the LNCaP human prostate cancer cell lines in 200 μL of Matrigel. Mice were divided into two groups, and the treatment group was implanted with 2 one cm long silastic implants filled with testosterone as described previously (6). Tumors were harvested 2- and 4-days post-treatment and fixed in 10% buffered formalin and processed for IHC and H&E staining. NK cells were identified using antiCD57-PE (Santa Cruz Biotechnology) and antiCD49b-FITC (Santa Cruz Biotechnology) antibodies. F4/80-Alexafluor 488 and Ly-6G-Alexafluor 488 antibodies (Biolegend) were used to stain the macrophages and neutrophils cells respectively. Stained sections were imaged using Zeiss LSM 700 laser confocal microscope. Images were analyzed using the image processing package Fiji. NK cells, macrophages and neutrophils were counted per field from at least five fields and plotted as mean values.

### 3.3.16 Patient Materials

The study was conducted in accordance with ethical guidelines as outlined by the Declaration of Helsinki. The Institutional Review Board approved this study at Johns Hopkins, and all accrued patients provided written informed consent. Biopsies from metastatic lesions were obtained from patients with castration-resistant prostate cancer, under a prospective protocol (clinicaltrials.gov: NCT03554317) examining the use of SupraT as a treatment for metastatic castration-resistant prostate cancer (mCRPC). These patients had previously been treated with standard androgen deprivation therapy as well as next-generation anti-androgen therapy using abiraterone and/or enzalutamide. Biopsies were typically obtained from soft-tissue metastatic sites (lymph nodes, liver, and lung) using an 18-gauge core biopsy needle and were collected before and 12 weeks after starting the SupraT treatment. For each metastatic biopsy, at least two fresh cores were collected for immediate flash-freezing in liquid nitrogen (Frozen tissue method), and at least two separate cores for formalin-fixed and paraffin-embedded sections (FFPE method). A dedicated tissue technician was called at the biopsy suite to initiate processing steps, including FFPE preparation within 30 minutes of biopsy collection. All samples were evaluated for adequacy and assessment of tumor-to-normal ratio by an expert urological pathologist. FFPE specimens were processed according to the standard procedures at the Johns Hopkins pathology department.

### 3.3.17 Immunohistochemical Staining and CD8 Density Measurement by Image Analysis

Chromogenic IHC for CD8 was performed as follows. Sections of 4-micron thickness were cut and placed on super frost plus slides. Paraffin sections were baked on a hot plate at 60° C. for 10 minutes, dewaxed using xylene, rehydrated in a series of graded alcohols to distilled water, and finally rinsed in distilled water with 0.1% Tween 20. Slides were transferred to a glass jar filled with a suitable antigen retrieval solution. The glass jar was irradiated in a microwave oven at full power for 1 minute, followed by 15 minutes at power level 20. Slides were cooled for 5 minutes at room temperature and washed 2× in tris-buffered saline with 0.1% Tween 20 (TBST). Tissues were subjected to endogenous peroxidase blocking using hydrogen peroxide for 5 minutes. Slides were then incubated with CD8 (DAKO, Clone: C8/144B) antibody for 45 minutes at room temperature, rinsed with TBST, and incubated with the secondary antibody (PowerVision Poly-HRP Anti-Mouse IgG, Leica, PV6119) for 30 minutes. Following incubation with the chromogen, 3-Amino-9-Ethylcarbazole (AEC) for 20 minutes, slides were counterstained with hematoxylin. After counterstaining, slides were washed with tap water for 2 minutes and distilled water for 1 minute and mounted using VectaMount AQ, Vector H-5501.

Whole biopsy slides stained for CD8 were scanned on a Roche-Ventana DP200 whole slide scanner and analyzed using the HALO 3.0 (Indica Labs) software. Regions of interest (ROI) consisted of tumor tissue that was delineated manually by a pathologist with expertise in prostate pathology. In cases with clear lymph node tissue from lymph node biopsies, regions were chosen to avoid encompassing the lymphoid tissue apart from the tumor. T cells were delineated using the cytonuclear IHC module in HALO. CD8 density was calculated as the number of T cells per mm² of ROI. Cell density measured using HALO were verified by manual counting in a subset of cases.

### 3.3.18 Statistical Analysis

Suitable central tendency values were calculated for all the quantitatively measurable variables and were used for the analysis of statistical significance. Parametric analysis was performed to compare mean values after estimating the normal distribution unless otherwise specified. These analyses were performed using Prism version 6.0 (GraphPad Software), and a value of p≤0.05 was considered significant in all the statistical analyses.

### 3.4 Results

Previous work demonstrated that sensitivity to SupraT differed among PCa cell lines (6). Cell cycle analysis revealed that SupraT causes a growth inhibitory accumulation of LNCaP cells in the G0/G1 phase, while LAPC4 cells show the opposite response and continue to proliferate. Furthermore, the clonogenic analysis revealed that SupraT significantly decreases the clonogenic potential of SupraT-sensitive LNCaP cells. To understand whether sensitivity to SupraT reflected DNA repair mutations in these cancer lines, we searched the COSMIC cell line database for mutations in DNA repair genes in PCa lines. Strikingly, our analysis revealed that sensitivity to hormone exposure correlated directly with the number of DNA repair mutations harbored by the cell types (LNCaP>VCaP>LAPC4), mirroring previously reported clinical data. Schweizer et al., 2015; Teply et al., 2017. 22R-v1, another AR positive line that is castration resistant, Sramkoski et al., 1999, was not inhibited by SupraT. Without wishing to be bound to any one particular theory, it is thought that due to the prevalence of DNA repair mutations, LNCaP and VCaP cells (the intermediate sensitivity line) treated with SupraT might not repair DSBs induced by androgens and instead undergo a DNA repair crisis leading to apoptosis. Contrary to this hypothesis, however, none of the prostate cancer cell lines treated with synthetic androgen R1881 had any substantial enhancement of PARP cleavage. We also measured Annexin-V positivity, an early marker of apoptosis, in the highly sensitive LNCaP cells and found no significant increase in Annexin-V after treatment. This observation suggested that the decrease in cell number upon testosterone treatment in the sensitive cell lines might likely also involve a nonapoptotic cell death mechanism. We, therefore, probed whether necrotic markers were induced upon SupraT treatment, and found them to be markedly reduced or unchanged, ruling out necrotic induction by SupraT. Recently, ferroptosis, a nonapoptotic cell death mechanism, has been linked to autophagy. Dixon et al., 2012; Stockwell et al., 2017. We first determined whether SupraT can induce autophagy in PCa lines. Treatment of PCa cell lines demonstrated that the synthetic androgen, R1881, notably induces autophagy in the SupraT sensitive LNCaP and VCaP cells, while basal autophagy in the SupraT-insensitive LAPC4 and 22Rv1 cells remained largely unaffected upon treatment. We did not find global upregulation of key autophagy proteins such as Beclin or ATG12. However, we did find both the number of autophagosomes and autophagy flux (evaluated using an autophagy flux sensor) increased only in LNCaP cells as compared to LAPC4 cells upon treatment with androgens. These results prompted us to determine whether SupraT is able to differentially induce ferroptosis in PCa cells that are sensitive to androgen.

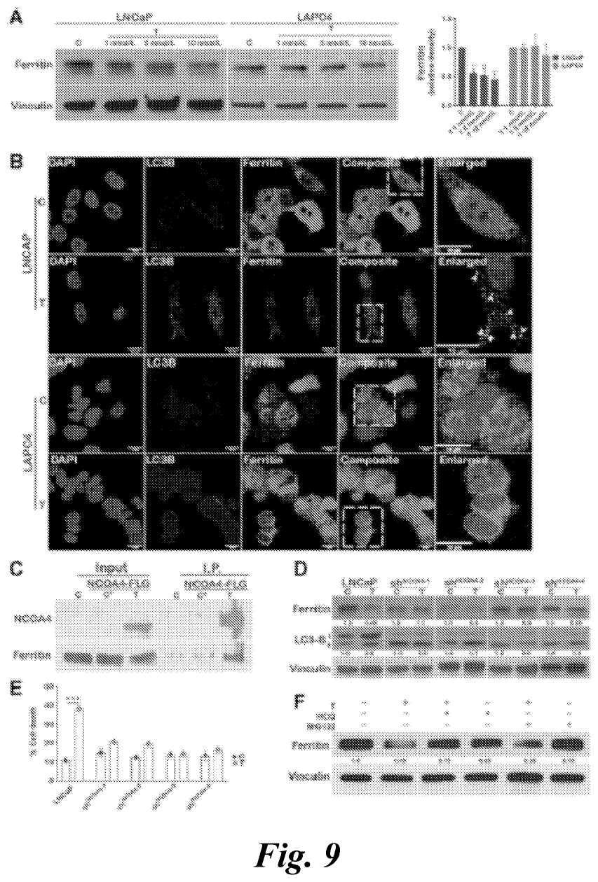

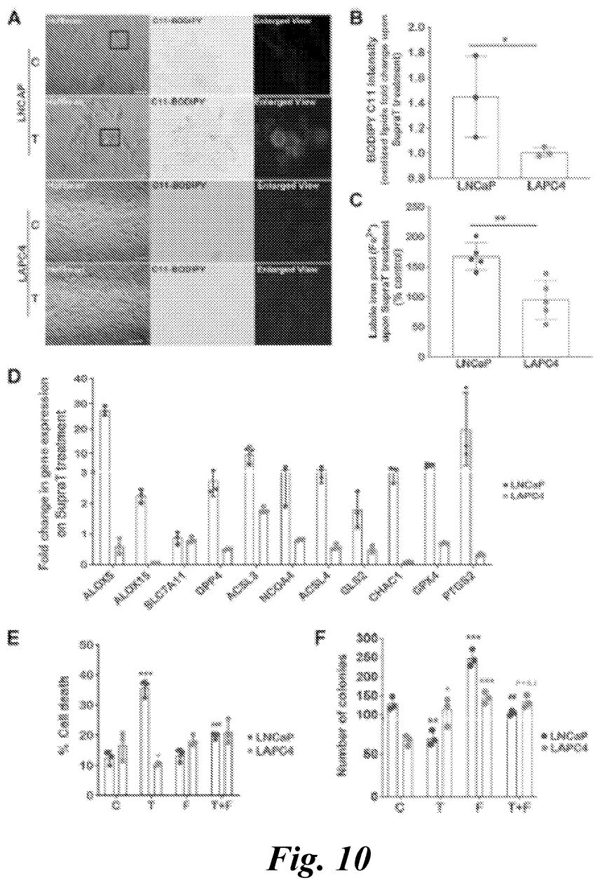

Ferroptosis involves iron-dependent accumulation of toxic lipid peroxides that leads to cell death. Dixon and Stockwell, 2014. Degradation of the iron storage protein ferritin through a specialized form of autophagy, termed ferritinophagy, increases the labile pool of iron, leading to an increase in lipid peroxides. Doll and Conrad, 2017. We sought to determine whether ferritinophagy is induced by SupraT. As depicted in FIG. 9A, SupraT causes a dose-dependent decrease in ferritin levels in LNCaP and VCaP cells compared to LAPC4 and 22Rv1 cells, where ferritin levels remain unchanged. We confirmed whether decrease in ferritin level is indeed through testosterone by treating cells with Dihydrotestosterone (DHT), a high affinity testosterone metabolite. Supraphysiological levels of DHT decreased ferritin levels in LNCaP cells but not in LAPC4 cells. Confocal images of SupraT treated LNCaP cells revealed that ferritin colocalized with LC3B positive autophagosomes (FIG. 9B). Since NCOA4 interacts with ferritin and mediates its autophagic degradation, Doll and Conrad, 2017; Dowdle et al., 2014; Mancias et al., 2014, we probed for NCOA4 and found that NCOA4 is induced by SupraT, and interacts with ferritin in a SupraT dependent manner (FIG. 9C). Further, knockdown of NCOA4 in LNCaP cells inhibited R1881 induced ferritin degradation and decreased cell death. (FIG. 9D, and FIG. 9E). Knockdown of NCOA4 also decreased SupraT induced autophagy (FIG. 9D). To determine whether ferritin is indeed degraded through autophagy, we treated cells with hydroxychloroquine, an autophagy inhibitor, and MG-132, a proteasome inhibitor, and evaluated its effect on ferritin degradation upon SupraT treatment. As seen in FIG. 9F, hydroxychloroquine prevented the degradation of ferritin by SupraT, suggesting that ferritin is degraded through autophagy. We evaluated the functional consequence of decreased ferritin on lipid peroxide formation using the lipid peroxide sensor, C11-BODIPY, Dixon and Stockwell, 2017; Naguib, 1998, which fluoresces green upon oxidation. As shown in FIG. 10A and FIG. 10B, R1881 treated LNCaP cells but not LAPC4 had increased lipid peroxides and labile iron pool (FIG. 10C) compared to vehicle treated controls. Next, we investigated whether SupraT induces pro-ferroptotic gene expression. Dixon et al., 2012; Friedmann et al., 2019. Pro-ferroptotic genes such as ALOX5, PTGS2, and NCOA4 were increased many folds over vehicle controls (FIG. 10D). We further confirmed that SupraT induced cell death in LNCaP is indeed through ferroptosis, as treatment with a combination of R1881 and a ferroptosis inhibitor, ferrostatin-1, abrogated cell death (FIG. 10E and FIG. 10F). A similar abrogation of SupraT induced cell death was observed in VCaP cells that demonstrates intermediate sensitivity to SupraT. Moreover, treatment of LNCaP and LAPC4 cells with DHT or a combination of DHT and ferrostatin-1 had the same effect.

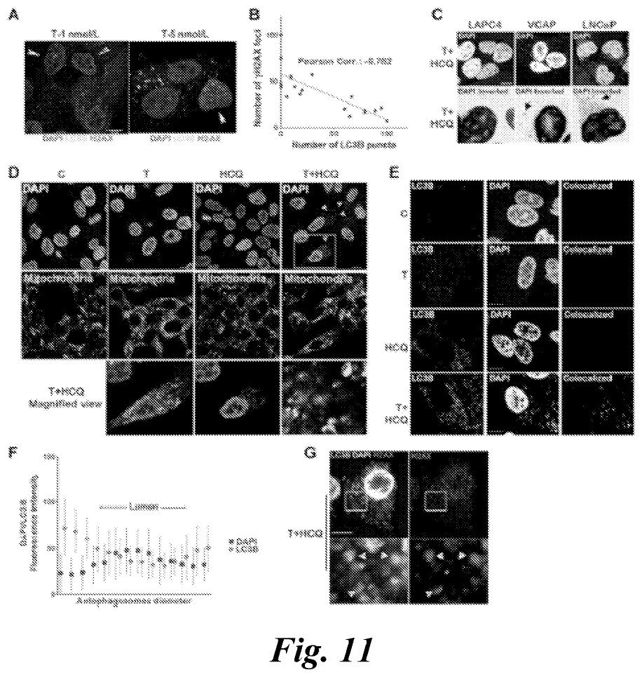

To investigate whether there is a direct link between response to SupraT, autophagy, and DNA repair, we treated SupraT sensitive LNCaP cells, which harbor several DNA repair gene mutations with R1881 and stained cells for autophagosomes and DSBs. We postulated that cells harboring damaged DNA would have increased autophagy in response to DNA damage stress. Interestingly, LNCaP cells with increased autophagosomes displayed a smaller number of γ-H2AX puncta (Pearson's correlation=−0.78) (FIG. 11A and FIG. 11B). This suggested that cells undergoing autophagy in response to SupraT might clear their damaged DNA more efficiently. Autophagy, being a dynamic event, renders visualization of fractions of autophagosomes carrying damaged DNA a challenge. Hydroxychloroquine prevents the fusion of autophagosomes to lysosomes, leading to accumulation of autophagosomes. Klionsky et al., 2016. Treatment of PCa cell lines with a combination of hydroxychloroquine and R1881 revealed marked localization of cytoplasmic DNA in LNCaP cells compared to the fewer cytoplasmic DNA puncta displayed in VCaP cells, which have intermediate DNA repair gene mutations. The SupraT-insensitive LAPC4 cells, however, did not show any cytoplasmic DNA (FIG. 11C). Intriguingly, although we did find induction of DSBs in SupraT treated LNCaP NCOA4 knockdown cells, similar to wild type cells, we did not find any cytoplasmic DNA in these cells after SupraT treatment. This could be as a result of abrogation of autophagy induction as evident from the number of autophagosomes between control and SupraT treated cells. To ensure that the observed cytoplasmic DNA was not mitochondrial DNA, we stained cells with antibodies against mitochondrial complex IV subunit I and LC3B. We did not find any notable co-localization of mitochondria with autophagosomes (FIG. 3D). However, we did find marked co-localization of cytoplasmic DAPI staining with LC3B in SupraT treated cells, indicating that the cytoplasmic DNA is present in autophagosomes (FIG. 11E). DAPI intensity peaked in the lumen of autophagosomes indicating that the DNA was indeed present within the autophagosomes (FIG. 11F). To ascertain whether the DNA within the autophagosomes harbored damaged DNA, we stained cells treated with a combination of hydroxychloroquine and SupraT with γ-H2AX and LC3B. As seen in FIG. 11G, LC3B and γ-H2AX colocalize with DAPI signals in the cytoplasm. This suggests that SupraT induced damaged DNA can be shuttled to the cytoplasm for autophagosome-mediated degradation. This implies that SupraT might induce two parallel autophagy-mediated phenomena: ferritinophagy and nucleophagy, both of which may be responsible for the growth inhibitory effects of SupraT.

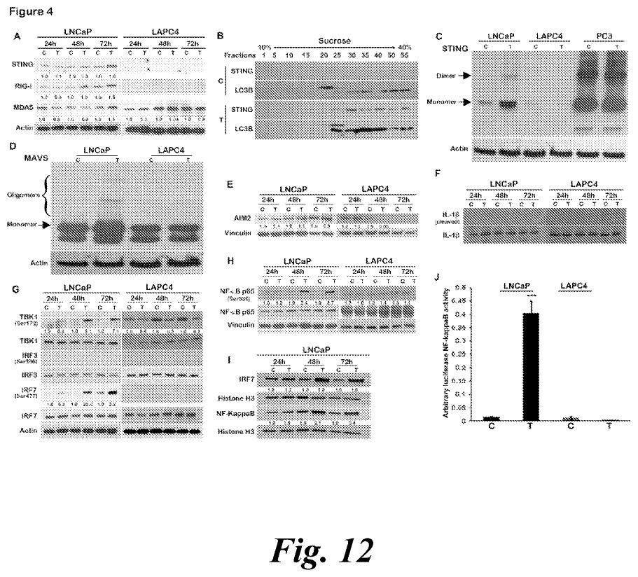

Cytosolic DNA is seen as a stimulant by the innate immune system as it is detected by DNA sensors in the cytoplasm that activate the adaptor protein STING and downstream innate immune signaling. Burdette and Vance, 2013; Yin et al., 2012. The STING promoter harbors an AR binding motif; however, we did not find any induction of the STING transcript or STING promoter by treatment with SupraT. Intriguingly, treatment with SupraT induced the STING protein as well as the RNA sensors RIG-I and MDA5 proteins in the SupraT sensitive LNCaP cells (FIG. 12A). Upon activation, monomeric STING dimerizes and translocates from the ER to autophagosome-like vesicles. Dobbs et al., 2015; Saitoh et al., 2009. Separation of cellular homogenates on sucrose gradients revealed STING cofractionates with LC3B positive autophagosomes in SupraT treated cells (FIG. 12B). To confirm that STING is indeed activated upon SupraT treatment, we performed a STING dimerization assay, a gold standard for STING activation. Yin et al., 2012. As shown in FIG. 12C, treatment with SupraT activates STING in LNCaP compared to LAPC4 cells. Interestingly, SupraT also activated MAVS oligomerization, indicating that the RNA sensing pathway was activated as well (FIG. 12D). Mislocalized and damaged DNA can also be detected by AIM2. Schroder et al., 2009. The AIM2 promoter harbors an AR binding motif; hence we first measured AIM2 transcript and protein levels. We did not find any induction of AIM2 promoter activity, AIM2 transcript levels or protein by SupraT (FIG. 12E). Neither did we find SupraT mediated activation of downstream inflammasomes, as evaluated by IL-1β cleavage, ruling out the involvement of AIM2 mediated inflammasome signaling by SupraT (FIG. 12F). We found a similar activation of STING, albeit less robustly, in VCaP cells. Interestingly in VCaP cells, STING appeared as a tetramer as reported for STING activation in some studies. Shang et al., 2019. We did not find any activation of STING in the SupraT insensitive 22Rv1 cells, neither did we find any activation of MAVS in both VCaP and 22Rv1 cells. We next investigated whether the observed effect is indeed due to testosterone by treating cells with DHT. In agreement with our findings using R1881, we found DHT robustly increased autophagy in LNCaP cells but not in LAPC4 cells. DHT also activated STING and MAVS preferentially in LNCaP cells, however, the activation was not as robust as R1881. This may be due to the fact that persistent H2AX foci are not observed in DHT treated cells as compared to R1881, Chatterjee et al., 2019, as DHT is rapidly metabolized. Brown et al., 1981; Kemppainen et al., 1992.

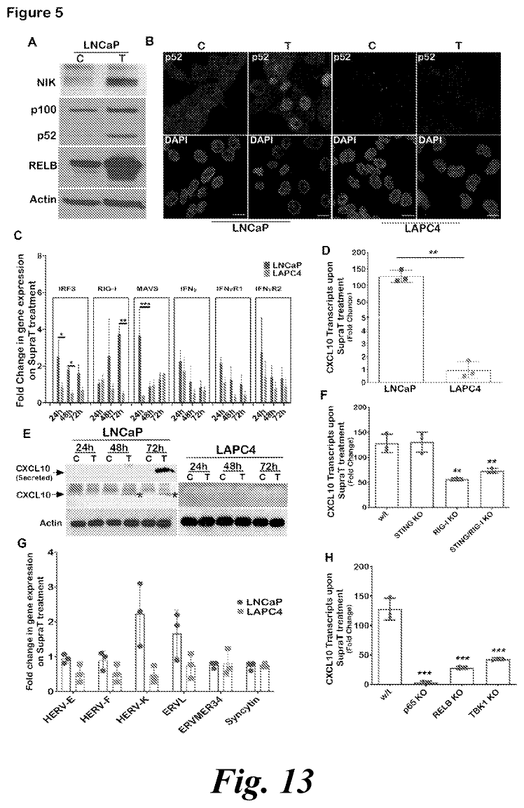

Following its own activation, STING activates the Tank binding kinase 1 (TBK1), which in turn phosphorylates and activates interferon regulatory factors (IRFs), including IRF3, IRF7, and NF-kappaB, leading to the induction of immune response genes. Burdette and Vance, 2013. Although we did not find any activation of IRF3 by SupraT treatment (FIG. 12G), our data indicates that TBK1, IRF7, and NF-kappaB canonical p65 subunit were activated upon SupraT treatment in LNCaP and VCaP cells (FIG. 12G and FIG. 12H). Activated p65 and IRF7 increased in the nucleus (FIG. 12I). Similar to LAPC4 cells, 22Rv1 cells did not show an induction of NF-kappaB or IRF7 signaling. We performed an NF-kappaB promoter-reporter assay, which confirmed the functional activation of NF-kappaB in SupraT sensitive cells (FIG. 12J). Intriguingly, we found SupraT stabilized NF-kappaB inducing kinase (NIK), a kinase central to the non-canonical NF-kappaB pathway (FIG. 13A). NIK phosphorylates the non-canonical p100 subunit of NF-kappaB and marks it for processing by a proteasome into the smaller p52 subunit, which subsequently dimerizes with RELB to activate transcription of target genes. Treatment with SupraT led to the formation of p52 and induction of RELB in the SupraT sensitive LNCaP cells but not in VCaP cells (FIG. 13A; probably reflecting differences in SupraT sensitivity of these cells. Immunofluorescence confirmed the nuclear translocation of p52 upon SupraT treatment (FIG. 13B). Further, in line with nucleic acid sensor activation, treatment with DHT led to stabilization of RelB and NIK and phosphorylation of the canonical and non-canonical NF-kappaB subunits downstream of nucleic acid sensors. This data indicates that SupraT induced DNA damage activates nucleic acid sensors and downstream NF-kappaB signaling.

To find out whether NF-kappaB responsive innate immune genes were activated by the nucleic acid sensors, we measured transcript levels of innate immune genes and found many genes were significantly upregulated in the SupraT-sensitive LNCaP and VCaP cell lines (FIG. 13C). CXCL10, a chemokine, was increased, Tokunaga et al., 2018, several folds in treated LNCaP cells both at transcript and protein level (FIG. 13D and FIG. 13E). CXCL10 was also induced (less robustly) in VCaP cells but not in SupraT insensitive 22Rv1 cells. Furthermore, DHT was able to induce CXCL10 (less robustly than R1881) preferentially in LNCaP cells as compared to LAPC4 cells. Bioplex assays confirmed the induction and secretion of CXCL10 and other chemokines upon SupraT treatment. To determine whether STING or RIG-I sensors play a role in the activation of downstream innate immune signaling, we created knockouts for STING, RIG-I, and STING/RIG-I double knockouts and tested whether they abrogate CXCL10 induction. Knockout of STING did not abrogate CXCL10 induction, but double knockouts and knockout of RIG-I alone decreased CXCL10 expression (FIG. 13F). This suggested that both the nucleic acid sensors are activated in SupraT-sensitive cells and that RIG-I may play an essential role in amplifying the signal. RIG-I can also be activated by the expression of endogenous retroviruses. Chiappinelli et al., 2015. Our analysis revealed that some of the endogenous retroviruses harbor androgen response elements. However, none of the endogenous retroviral transcripts investigated were upregulated by SupraT (FIG. 13G), suggesting activation of RIG-I may be primarily through cytoplasmic DNA, as indicated by others. Ablasser et al., 2009.

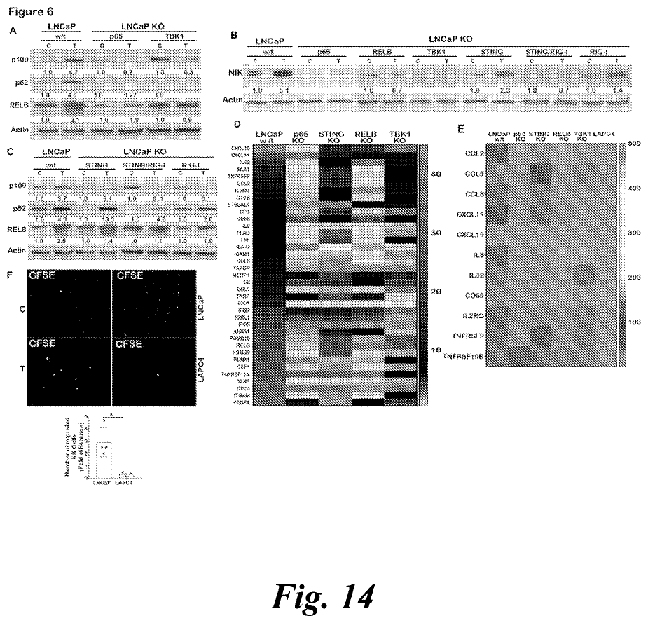

To tease out whether the canonical or the non-canonical NF-kappaB pathway is important for innate immune gene signaling by SupraT, we made knockouts of TBK1 and other key components of the NF-kappaB pathway. Knockouts of p65, TBK1, and RELB each abrogated downstream CXCL10 induction by SupraT without altering the growth inhibitory effect of SupraT on of these cells (FIG. 13H). Induction or proteolytic processing of non-canonical subunits RELB and p100, respectively, by SupraT was totally abrogated in p65 and TBK1 knockouts (FIG. 14A), indicating that the canonical pathway is important for the activation of the non-canonical pathway and TBK1 may play a critical role in linking the two. Furthermore, knockouts of p65, RELB, and TBK1 each abrogated NIK stabilization compared to parental cells, knockouts of STING and RIG-I decreased NIK stabilization, and STING/RIG-I double knockouts totally diminished the NIK stabilization, mirroring that of the p65 and TBK1 knockouts (FIG. 14B). Intriguingly, knockouts of STING did not revoke induction or processing of p100, but knockouts of RIG-I alone decreased p100 induction. Further, STING/RIG-I double knockouts had lower induction of RELB and p100 processing as compared to STING knockouts (FIG. 14C). While this data corroborates the involvement of both the nucleic acid sensors, STING and RIG-I may have opposite effects on the non-canonical NF-kappaB signaling induced by SupraT-STING being suppressive and RIG-I being supportive.

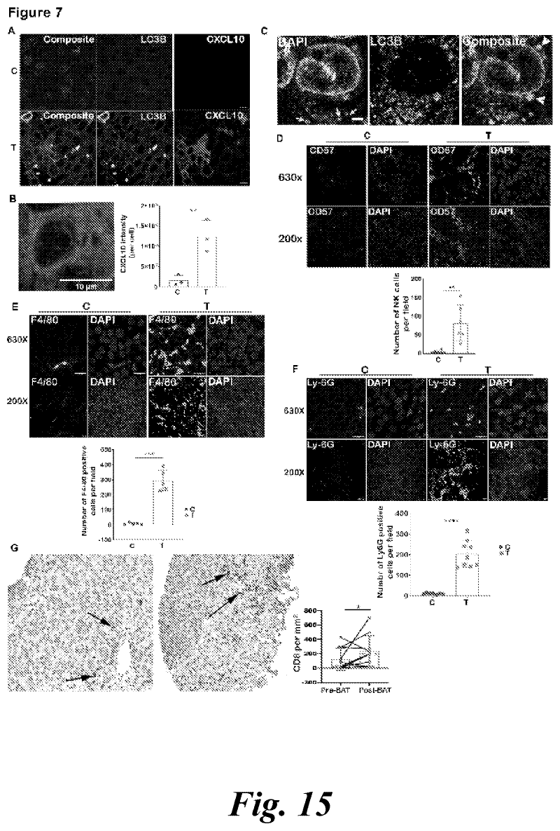

To identify immune genes activated by SupraT and discern those regulated by the STING-TBK1-NFkappaB axis, we performed a Nanostring PanCancer IO 360 Gene Expression analysis using a panel of 770 unique immune-gene signatures. Out of 57, differentially (>2.5 fold) expressed genes, 38 percent of immune genes were induced in a STING-TBK1-NFkappaB dependent manner (FIG. 14D). We validated a panel of chemokines and cytokines that play a role in attracting immune cells using quantitative PCR, and in concurrence with our Nanostring data, these genes were altered in a STING-TBK1-NFkappaB dependent manner (FIG. 14E). The gene expression data further indicates that both innate and adaptive immune cells might home to and get activated by SupraT induced cytokines and chemokines. To investigate whether SupraT is able to induce migration of NK cells, we conducted a transwell migration assay with human NK-92 cells and found the SupraT-sensitive cell line significantly increased migration of NK-92 cells upon treatment (FIG. 14F). We next established LNCaP xenografts in athymic nude mice, which exhibit robust NK cell activation and harbor neutrophils and macrophages. Sheil et al., 1984. In concordance with in vitro findings, SupraT led to an increase in autophagosomes, the presence of cytoplasmic DNA, and an increase in CXCL10 expression by tumor cells (FIG. 15A and FIG. 15B). The presence of autophagosomal DNA without any autophagy inhibition indicated that the dynamics of autophagosomal degradation differed in vitro and in vivo (FIG. 15C). Staining for NK cell markers CD57 and CD49b revealed a 17-fold induction in NK cell migration to the tumor bed (FIG. 15D). A similar result was obtained when tumors were stained for macrophages and neutrophils (FIG. 15E and FIG. 15F). Prostate tumors are considered immunologically cold tumors with limited cytotoxic T cell infiltration. Fong et al., 2014. The clinical significance of our data was investigated using biopsy from patients undergoing BAT therapy. As seen in FIG. 15G, BAT administration significantly increased the infiltration of CD8 T cells. In summary, these results indicate that SupraT may be able to activate immune cells through the NF-kappaB pathway by activating nucleic acid sensors, especially in cells with defects in DNA repair pathways. Our data also indicates that SupraT may induce ferroptosis, a potentially immunogenic cell death mechanism.

### 3.5 Discussion

Strategies to overcome resistance to ADT can make a significant impact on the current outcomes of therapy. Several complementary mechanisms for the paradoxical effect of SupraT on PCa have been described, including cellular senescence and cell death. Vander et al., 2007; Roediger et al., 2014; Wen et al., 2014; Isaacs, 1984. Understanding how BAT works at the molecular and cellular levels might help in rationally combining BAT with other agents to achieve increased efficacy and tumor response. Our findings that the administration of SupraT may lead to ferroptosis mediated by lipid peroxides is intriguing. It is speculated that PCa cells rely on lipid metabolism for their growth, and targeting lipid metabolism to overcome prostate cancer growth is viewed as a possible therapeutic strategy. Schlaepfer et al., 2014. SupraT may also influence both lipid uptake and synthesis, as androgens have been linked to regulating lipid metabolism. Swinnen et al., 2006; Suburu and Chen, 2012; Butler et al., 2016. The increased cellular lipids may serve as substrates for the generation of lipid peroxides, leading to ferroptosis. Future work will provide more insights into the role of SupraT induced lipid metabolism in ferroptosis.

Based on our previous experimental and clinical reports that SupraT induces DSBs and extreme responders to BAT therapy harbor DNA repair mutations, Schweizer et al., 2015; Teply et al., 2017; Teply et al., 2018, we speculated that PCa harboring mutations in the DSB repair pathway would be sensitive to SupraT. We were surprised to note that the damaged DNA was shuttled for autophagosomal degradation. Such nucleophagic degradation of damaged DNA has been noted for radiation- and chemo-therapy. Park et al., 2009; Eapen et al., 2017; Deng et al., 2014. A key finding of our study is that SupraT induces STING and downstream NF-kappaB-driven immune genes to activate immune infiltration in vitro, in animal xenografts, and in patient resected tumors. Our results further provide insights into cross-talk between the canonical and non-canonical NF-kappaB pathways induced by SupraT. Previous studies have shown that the canonical pathway is rapidly induced upon stimulation, while chronic stimulation is required for the non-canonical pathway to become activated. Sun, 2017. These results indicate that the canonical pathway is induced forty-eight hours after SupraT administration, which synchronizes with the induction of autophagy. This early induction suggests that the activation of the canonical pathway might not be a direct response to SupraT, but maybe an indirect response to SupraT-mediated nucleophagy. A key question that remains to be addressed is whether autophagy induction occurs in response to the presence of damaged DNA in the cytoplasm or is induced by SupraT irrespective of DNA repair competency. We did not detect a global increase in autophagic proteins like Beclin or ATG12. Moreover, autophagy was not induced in AR-positive SupraT-insensitive LAPC4 or 22Rv1 cells, indicating that autophagy induction may not solely depend on AR status or SupraT treatment. A decrease in autophagy induction upon NCOA4 knockdown in SupraT treated cells indicated that NCOA4 may play an important role SupraT mediated autophagy-a feature that requires further investigation. Another noteworthy observation was the induction of both the STING and RIG-I pathways. While STING activation is mediated by non-self and damaged-self cytoplasmic DNA, RIG-I is stimulated by 5′-triphosphorylated short double-stranded RNA. Rehwinkel and Gack, 2020. Through mechanisms not yet fully understood, RIG-I is also activated by double-stranded DNA. Ablasser et al., 2009. We found MDA5, another RNA sensor, was also induced by SupraT. While the contributions of MDA5 to SupraT mediated innate immune signaling remain to be elucidated, it is likely to play a minor role as double knockouts of STING/RIG-I were sufficient to abrogate NIK stabilization and NF-kappaB signaling.

STING signaling is considered a double-edged sword as chronic STING signaling is considered pro-tumorigenic, while acute STING signaling is considered anti-tumorigenic. Kwon and Bakhoum, 2020. Our data indicates that STING is acutely induced by SupraT, a feature that may contribute to its anti-tumor effect. Infiltration of immune cells in tumor xenografts and patient biopsies further indicates that the immune system is engaged upon SupraT administration. This observation has clinical significance as the presence of immune infiltrates is a key parameter and correlates with therapeutic response to immunotherapy. Zou et al., 2016. Recently, immune checkpoint therapy has been shown to induce ferroptosis in tumor cells. Wang et al., 2019. Ferroptosis itself may likely potentiate the immune clearance of tumors through the release of damage-associated molecular patterns. Friedmann et al., 2019. Understanding the role of ferroptosis in SupraT induced immune signaling, may provide additional mechanistic insights into cellular immune response. Further, cytokines and chemokine genes induced by the STING-TBK1-NF-kappaB pathway may serve as predictors of therapeutic response in the future. Impending clinical investigations involving the combination of SupraT with immune checkpoint inhibitors may be further informative.

In summary, our findings suggests that SupraT induces two autophagy-mediated pathways, namely ferritinophagy and nucleophagy. While ferritinophagy may induce ferroptosis, consequently, leading to cell death; nucleophagy induces innate immune signaling (through nucleic acid sensing-NF kappaB signaling) and infiltration of immune cells.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Ablasser A, Bauernfeind F, Hartmann G, Latz E, Fitzgerald K A, and Hornung V. RIG-I-dependent sensing of poly(dA:dT) through the induction of an RNA polymerase III-transcribed RNA intermediate. Nat Immunol. 2009; 10 (10): 1065-72.
Antonarakis E S, et al., AR-V7 and resistance to Enzalutamide and Abiraterone in prostate cancer. N Engl J Med. 2014; 371:1028-1038.
Balbas M D, Evans M J, Hosfield D J, Wongvipat J, Arora V K, Watson P A, Chen Y, Greene G L, Shen Y, Sawyers C L. Overcoming mutation-based resistance to antiandrogens with rational drug design. Elife. 2013 Apr. 9; 2: e00499.
Beer T M, Armstrong A J, Rathkopf D E, Loriot Y, Sternberg C N, Higano C S, Iversen P, Bhattacharya S, Carles J, Chowdhury S, Davis I D, de Bono J S, Evans C P, Fizazi K, Joshua A M, Kim C S, Kimura G, Mainwaring P, Mansbach H, Miller K, Noonberg S B, Perabo F, Phung D, Saad F, Scher H I, Taplin M E, Venner P M, Tombal B; the PREVAIL Investigators. Enzalutamide in Metastatic Prostate Cancer before Chemotherapy. N Engl J Med. 2014 Jun. 1. [Epub ahead of print]
Behre H M, Nieschlag E. Testosterone preparations for clinical use in males. In: Testosterone: Action, Deficiency, Substitution 4th Edition (E Nieschlag, H. M. Behre, eds.) Cambridge University Press, Cambridge UK, 2012, pp. 309-35.
Beltran H, Yelensky R, Frampton G M, Park K, Downing S R, MacDonald T Y, Jarosz M, Lipson D, Tagawa S T, Nanus D M, Stephens P J, Mosquera J M, Cronin M T, Rubin M A. Targeted next-generation sequencing of advanced prostate cancer identifies potential therapeutic targets and disease heterogeneity. Eur Urol. 2013; 63:920-6.
Bergerat J, Ceraline J. Pleiotropic functional properties of androgen receptor mutants in prostate cancer. Hum Mutat. 2009; 30:145-57.
Bianchini D, Lorente D, Rodriguez-Vida A, Omlin A, Pezaro C, Ferraldeschi R, Zivi A, Attard G, Chowdhury S, de Bono J S. Antitumour activity of enzalutamide (MDV3100) in patients with metastatic castration resistant prostate cancer (CRPC) pre-treated with docetaxel and abiraterone. Eur J Cancer. 2013 Sep. 25. [Epub ahead of print]
Brendler H, Chase W E, Scott W W. Prostatic Cancer: Further investigation of hormone relationships. Arch Surg 1949; 433-440.
Brown T R, Rothwell S W, and Migeon C J. Comparison of methyltrienolone and dihydrotestosterone binding and metabolism in human genital skin fibroblasts. J Steroid Biochem. 1981; 14 (10): 1013-22.
Burdette D L, and Vance R E. STING and the innate immune response to nucleic acids in the cytosol. Nat Immunol. 2013; 14 (1): 19-26.
Butler L M, Centenera M M, and Swinnen J V. Androgen control of lipid metabolism in prostate cancer: novel insights and future applications. Endocr Relat Cancer. 2016; 23 (5): R219-27.
Chatterjee P, Schweizer M T, Lucas J M, Coleman I, Nyquist M D, Frank S B, Tharakan R, Mostaghel E, Luo J, Pritchard C C, et al. Supraphysiological androgens suppress prostate cancer growth through androgen receptor-mediated DNA damage. J Clin Invest. 2019; 129 (10): 4245-60.
Chen C D, Welsbie D S, Tran C, Baek S H, Vessella R, Rosenfeld M G, Sawyers C L. Molecular determinants of resistance to antiandrogen therapy. Nat Med 2004; 10:33-9.
Chen Y, Pullambhatla M, Byun Y, et al. 2-(3-{1-Carboxy-5-[(6-[18F]fluoro-pyridine-3-carbonyl)-amino]-pentyl}-urei do)-pentanedioic acid, [18F]DCFPyL, a PSMA-based PET Imaging Agent for Prostate Cancer. Clin Cancer Res. Oct. 31, 2011.
Chiappinelli K B, Strissel P L, Desrichard A, Li H, Henke C, Akman B, Hein A, Rote N S, Cope L M, Snyder A, et al. Inhibiting DNA Methylation Causes an Interferon Response in Cancer via dsRNA Including Endogenous Retroviruses. Cell. 2015; 162 (5): 974-86.
Cho, S. Y., Gage, K. L., Mease, R. C., Senthamizhchelvan, S., Holt, D. P., Jeffrey-Kwanisai, A., Endres, C. J., Dannals, R. F., Sgouros, G., Lodge, M., Eisenberger, M. A., Rodriguez, R., Carducci, M. A., Rojas, C., Slusher, B. S., Kozikowski, A. P., and Pomper, M. G. Biodistribution, tumor detection, and radiation dosimetry of 18F-18F-DCFPyL, a low-molecular-weight inhibitor of prostate-specific membrane antigen, in patients with metastatic prostate cancer. J Nucl Med 2012; 53; 1883-1891.
Crawford E D, Eisenberger M A, McLeod D G, Spaulding J T, Benson R, Dorr F A, Blumenstein B A, Davis M A, Goodman P J. A controlled trial of leuprolide with and without flutamide in prostatic carcinoma. N Engl J Med. 1989; 321:419-24.
de Bono J S, Logothetis C J, Molina A, Fizaki K, North S, Chu L, Chi K N, Jones R J, Goodman O B, Saad F, Staffurth J N, Mainwaring P, Harland S, Flaig T W, Hutson T E, Cheng T, Patterson H, Hainsworth J D, Ryan C J, Sternberg C N, Ellard S L, Flechon A, Saleh M, Scholz M, Efstathiou E, Zivi A, Bianchini D, Loriot Y, Chieffo N, Kheoh T, Haqq C M, Scher H I, Cou-AA-301 Investigators. Abiraterone and increased survival in metastatic prostate cancer. N Engl J Med 2011; 364:1995-2005.
Dehm S M, Schmidt L J, Heemers H V, Vessella R L, Tindall D J. Splicing of a novel androgen receptor exon generates a constitutively active androgen receptor that mediates prostate cancer therapy resistance. Cancer Res. 2008; 68:5469-77.
Deng L, Liang H, Xu M, Yang X, Burnette B, Arina A, Li X D, Mauceri H, Beckett M, Darga T, et al. STING-Dependent Cytosolic DNA Sensing Promotes Radiation-Induced Type I Interferon-Dependent Antitumor Immunity in Immunogenic Tumors. Immunity. 2014; 41 (5): 843-52.
Denmeade S R, et al. TRANSFORMER: A Randomized Phase II Study Comparing Bipolar Androgen Therapy vs. Enzalutamide in Asymptomatic Men with Castration Resistant Metastatic Prostate Cancer. J Clin Onc. In Press, 2020.
Denmeade S R, Isaacs J T. Bipolar androgen therapy: The rationale for rapid cycling of supraphysiologic androgen/ablation in men with castration resistant prostate cancer. Prostate. 2010; 70:1600-7.
Denmeade S R. Bipolar androgen therapy in the treatment of prostate cancer. Clin Adv Hematol Oncol. 2018; 16:408-411.
Dixon S J, and Stockwell B R. The role of iron and reactive oxygen species in cell death. Nat Chem Biol. 2014; 10 (1): 9-17.
Dixon S J, Lemberg K M, Lamprecht M R, Skouta R, Zaitsev E M, Gleason C E, Patel D N, Bauer A J, Cantley A M, Yang W S, et al. Ferroptosis: an iron-dependent form of nonapoptotic cell death. Cell. 2012; 149 (5): 1060-72.
Dobbs N, Burnaevskiy N, Chen D, Gonugunta V K, Alto N M, and Yan N. STING Activation by Translocation from the ER Is Associated with Infection and Autoinflammatory Disease. Cell Host Microbe. 2015; 18 (2): 157-68.
Doll S, and Conrad M. Iron and ferroptosis: A still ill-defined liaison. IUBMB Life. 2017; 69 (6): 423-34.
Donati R M, Ellis H, Gallagher N I. Testosterone potentiated 32P therapy in prostatic carcinoma. Cancer 1966; 19:1088-90.
Dowdle W E, Nyfeler B, Nagel J, Elling R A, Liu S, Triantafellow E, Menon S, Wang Z, Honda A, Pardee G, et al. Selective VPS34 inhibitor blocks autophagy and uncovers a role for NCOA4 in ferritin degradation and iron homeostasis in vivo. Nat Cell Biol. 2014; 16 (11): 1069-79.
Dressman D, Yan H, Traverso G, Kinzler K W, Vogelstein B. Transforming single DNA molecules into fluorescent magnetic particles for detection and enumeration of genetic variations. Proc Natl Acad Sci USA 2003; 100:8817-22.
Eapen V V, Waterman D P, Bernard A, Schiffmann N, Sayas E, Kamber R, Lemos B, Memisoglu G, Ang J, Mazella A, et al. A pathway of targeted autophagy is induced by DNA damage in budding yeast. Proc Natl Acad Sci USA. 2017; 114 (7): E1158-E67.
Fong L, Carroll P, Weinberg V, Chan S, Lewis J, Corman J, Amling C L, Stephenson R A, Simko J, Sheikh N A, et al. Activated lymphocyte recruitment into the tumor microenvironment following preoperative sipuleucel-T for localized prostate cancer. J Natl Cancer Inst. 2014; 106 (11).
Friedmann Angeli J P, Krysko D V, and Conrad M. Ferroptosis at the crossroads of cancer-acquired drug resistance and immune evasion. Nat Rev Cancer. 2019; 19 (7): 405-14.
Ghosh A, Heston W D. Tumor target prostate specific membrane antigen (PSMA) and its regulation in prostate cancer. J Cell Biochem. 2004; 91:528-39.
Haapala K, Hyytinen E R, Roiha M, Laurila M, Rantala I, Helin H J, et al. Androgen receptor alterations in prostate cancer relapsed during a combined androgen blockade by orchiectomy and bicalutamide. Lab Invest 2001; 81:1647.
Haffner, M C, Aryree M J, Toubaji A, Esopi D M, Albadine R, Gurel B, Isaacs W B, Bova G S, Liu W, Xu J, Meeker A K, Netto G, De Marzo A M, Nelson W G, Yegnasubramanian S. Androgen-induced TOP2Bmediated double-strand breaks and prostate cancer gene rearrangements. Nat Genet 2010; 42:668-75.
Hara T, Miyazaki J, Araki H, Yamaoka M, Kanzaki N, Kusaka M, et al. Novel mutations of androgen receptor: a possible mechanism of bicalutamide withdrawal syndrome. Cancer Res 2003; 63:149.
Harris W P, Mostaghel E A, Nelson P S, and Montgomery B. Androgen deprivation therapy: progress in understanding mechanisms of resistance and optimizing androgen depletion. Nature clinical practice Urology. 2009; 6 (2): 76-85.
Hu R, Denmeade S R, Luo J. Molecular processes leading to aberrant androgen receptor signaling and castration resistance in prostate cancer. Expert Rev Endocrinol Metab. 2010; 5:753-764.
Hu R, Dunn T A, Wei S, Isharwal S, Veltri R W, Humphreys E, Han M, Partin A W, Vessella R L, Isaacs W B, Bova G S, Luo J. Ligand-independent androgen receptor variants derived from splicing of cryptic exons signify hormone-refractory prostate cancer. Cancer Res. 2009; 69:16-22.
Hu R, Lu C, Mostaghel E A, Yegnasubramanian S, Gurel M, Tannahill C, Edwards J, Isaacs W, Nelson P S, Bluemn E, Plymate S R, Luo J. Distinct transcriptional programs mediated by the ligand-dependent full length androgen receptor and its splice variants in castration-resistant prostate cancer. Cancer Res. 2012; 72:3457-62.
Huggins C, Hodges C V. Studies on prostatic cancer. I. The effect of castration, of estrogen and androgen injection on serum phosphatases in metastatic carcinoma of the prostate. Cancer Research. 1941; 1 (293-7.
Hussain M H, Pienta K J, Redman B G, Cummings G D, Flaherty L E. Oral etoposide in the treatment of hormone-refractory prostate cancer. Cancer. 1994; 74:100-3.
Isaacs J T, Brennen W N, Denmeade S R. Rationale for bipolar androgen therapy (BAT) for metastatic prostate cancer. Cell Cycle. 2017 Aug. 18:1-2.
Isaacs J T, Brennen W N, Denmeade S R. Serial bipolar androgen therapy (sBAT) using cyclic supraphysiologic testosterone (STP) to treat metastatic castration-resistant prostate cancer (mCRPC). Ann Transl Med. 2019 December; 7 (Suppl 8): S311.
Isaacs J T, D'Antonio J M, Chen S, Antony L, Dalrymple S P, Ndikuyeze G H, Luo J, and Denmeade S R. Adaptive auto-regulation of androgen receptor provides a paradigm shifting rationale for bipolar androgen therapy (BAT) for castrate resistant human prostate cancer. Prostate. 2012; 72 (14): 1491-505.
Isaacs J T. Antagonistic effect of androgen on prostatic cell death. Prostate. 1984; 5 (5): 545-57.
Jemal A, Siegel R, Ward E, Hao Y, Xu J, Murray T, Thun M J. Cancer statistics, 2008. CA Cancer J Clin. 2008; 58:71-96.
Jennison C, Turnbull B W. Sequential equivalence testing and repeated confidence intervals, with applications to normal and binary responses. Biometrics. 1993; 49:31-43.
Jiang Y, Palma J F, Agus D B, Wang Y, Gross M E. Detection of androgen receptor mutations in circulating tumor cells in castration-resistant prostate cancer. Clin Chem. 2010; 56:1492-5.
Johnson D E, Haynie T P. Phosphorus-32 for intractable pain in carcinoma of prostate: Analysis of androgen priming, parathormone rebound, and combination therapy. Urology 1977; 9:137-39.
Joseph J D, Lu N, Qian J, Sensintaffar J, Shao G, Brigham D, Moon M, Maneval E C, Chen I, Darimont B, Hager J H. A Clinically Relevant Androgen Receptor Mutation Confers Resistance to Second-Generation Antiandrogens Enzalutamide and ARN-509. Cancer Discov. 2013; 3:1020-9.
Kemppainen J A, Lane M V, Sar M, and Wilson E M. Androgen receptor phosphorylation, turnover, nuclear transport, and transcriptional activation. Specificity for steroids and antihormones. J Biol Chem. 1992; 267 (2): 968-74.
Kinde I, Papadopoulos N, Kinzler K W, Vogelstein B. FAST-SeqS: a simple and efficient method for the detection of aneuploidy by massively parallel sequencing. PLOS One. 2012; 7: e41162.
Klionsky D J, Abdelmohsen K, Abe A, Abedin M J, Abeliovich H, Acevedo Arozena A, Adachi H, Adams C M, Adams P D, Adeli K, et al. Guidelines for the use and interpretation of assays for monitoring autophagy (3rd edition). Autophagy. 2016; 12 (1): 1-222.
Korpal M, Korn J M, Gao X, Rakiec D P, Ruddy D A, Doshi S, Yuan J, Kovats S G, Kim S, Cooke V G, Monahan J E, Stegmeier F, Roberts T M, Sellers W R, Zhou W, Zhu P. An F876L mutation in androgen receptor confers genetic and phenotypic resistance to MDV3100 (Enzalutamide). Cancer Discov. 2013; 3:1030-43.
Kwon J, and Bakhoum S F. The Cytosolic DNA-Sensing cGAS-STING Pathway in Cancer. Cancer Discov. 2020; 10 (1): 26-39.
Lam H M, Nguyen H M, Labrecque M P, Brown L G, Coleman I M, Gulati R, Lakely B, Sondheim D, Chatterjee P, Marck B T, et al. Durable Response of Enzalutamide-resistant Prostate Cancer to Supraphysiological Testosterone Is Associated with a Multifaceted Growth Suppression and Impaired DNA Damage Response Transcriptomic Program in Patient-derived Xenografts. Eur Urol. 2020; 77 (2): 144-55.
Laufer M, Denmeade S R, Sinibaldi V J, Carducci M A, Eisenberger M A. Complete androgen blockade for prostate cancer: what went wrong? J Urol. 2000; 164:3-9.
Linja M J, Savinainen K J, Saramaki O R, Tammela T L, Vessella R L, and Visakorpi T. Amplification and overexpression of androgen receptor gene in hormone-refractory prostate cancer. Cancer Res. 2001; 61 (9): 3550-5.
Litvinov I V, Vander Griend D J, Antony L, Dalrymple S, De Marzo A M, Drake C C, Isaacs J T. Androgen receptor as a licensing factor for DNA replication in androgen-sensitive prostate cancer cells. Proc Natl Acad Sci 2006; 103:15085-90.
Loriot Y, et al. Antitumour activity of abiraterone acetate against metastatic castration-resistant prostate cancer progressing after docetaxel and enzalutamide (MDV3100). Ann Oncol 2013; 24:1807-12.
Mancias J D, Wang X, Gygi S P, Harper J W, and Kimmelman A C. Quantitative proteomics identifies NCOA4 as the cargo receptor mediating ferritinophagy. Nature. 2014; 509 (7498): 105-9.
Mani R S, Tomlins S A, Callahan K, Ghosh A, Nyati M K, Varambally S, Palanisamy N, and Chinnaiyan A M. Induced chromosomal proximity and gene fusions in prostate cancer. Science. 2009; 326 (5957): 1230.
Manni A, Bartholomew M, Caplan R, Boucher A, Santen R, et al. Androgen Priming and Chemotherapy in Advanced Prostate Cancer: Evaluation of Determinants of Clinical Outcome. J Clin Oncol 1988; 6:1456-1466
Mark C. Markowski S K, Angelo M De Marzo, Samuel R. Denmeade, Emmanuel S. Antonarakis. Molecular and clinical characterization of metastatic castration-resistant prostate cancer (mCRPC) patients achieving deep PSA responses to bipolar androgen therapy (BAT). J Clin Oncol. 2021; 39 (
Markowski M C, Shenderov E, Eisenberger M A, Kachhap S, Pardoll D M, Denmeade S R, Antonarakis E S. Extreme responses to immune checkpoint blockade following bipolar androgen therapy and enzalutamide in patients with metastatic castration resistant prostate cancer. Prostate. 2020 Jan. 23.
Markowski M C, Wang H, Sullivan R, Rifkind I, Sinibaldi V, Schweizer M T, Teply B A, Ngomba N, Fu W, Carducci M A, Paller C J, Marshall C H, Eisenberger M A, Luo J, Antonarakis E S, Denmeade S R. A Multicohort Open-label Phase II Trial of Bipolar Androgen Therapy in Men with Metastatic Castration-resistant Prostate Cancer (RESTORE): A Comparison of Post-abiraterone Versus Post-enzalutamide Cohorts. Eur Urol. 2020 Jul. 2: S0302-2838 (20) 30471-1.
Morris M J, Huang D, Kelly W K, Slovin S F, Stephenson R D, Eicher C, Delacruz A, Curley T, Schwartz L H, Scher H I. Phase 1 trial of high-dose exogenous testosterone in patients with castration-resistant metastatic prostate cancer. Eur Urol. 2009 August; 56 (2): 237-44.
Naguib Y M. A fluorometric method for measurement of peroxyl radical scavenging activities of lipophilic antioxidants. Anal Biochem. 1998; 265 (2): 290-8.
Noonan K L, North S, Bitting R L, Armstrong A J, Ellard S L, Chi K N. Clinical activity of abiraterone acetate in patients with metastatic castration-resistant prostate cancer progressing after enzalutamide. Ann Oncol 2013; 24:1802-7.
Noss K R, Wolfe S A, Grimes S R. Upregulation of prostate specific membrane antigen/folate hydrolase transcription by an enhancer. Gene. 2002 Feb. 20; 285 (1-2): 247-56.
Park Y E, Hayashi Y K, Bonne G, Arimura T, Noguchi S, Nonaka I, and Nishino I. Autophagic degradation of nuclear components in mammalian cells. Autophagy. 2009; 5 (6): 795-804.
Prout G R, Brewer W R. Response of men with advanced prostatic carcinoma to exogenous administration of testosterone. Cancer 1967; 20:1871-1878.
Rehwinkel J, and Gack M U. RIG-I-like receptors: their regulation and roles in RNA sensing. Nat Rev Immunol. 2020; 20 (9): 537-51.
Robitaille A C, Mariani M K, Fortin A, and Grandvaux N. A High Resolution Method to Monitor Phosphorylation-dependent Activation of IRF3. J Vis Exp. 2016107): e53723.
Roediger J, Hessenkemper W, Bartsch S, Manvelyan M, Huettner S S, Liehr T, Esmaeili M, Foller S, Petersen I, Grimm M O, et al. Supraphysiological androgen levels induce cellular senescence in human prostate cancer cells through the Src-Akt pathway. Mol Cancer. 2014; 13 (214.
Ryan C J, Smith M R, de Bono J S, Molina A, Logothetis C J, de Souza P, Fizazi K, Mainwaring P, Piulats J M, Ng S, Carles J, Mulders P F, Basch E, Small E J, Saad F, Schrijvers D, Van Poppel H, Mukherjee S D, Suttmann H, Gerritsen W R, Flaig T W, George D J, Yu E Y, Efstathiou E, Pantuck A, Winquist E, Higano C S, Taplin M E, Park Y, Kheoh T, Griffin T, Scher H I, Rathkopf D E; COU-AA-302 Investigators. Abiraterone in metastatic prostate cancer without previous chemotherapy. N Engl J Med. 2013; 368:138-48.
Saitoh T, Fujita N, Hayashi T, Takahara K, Satoh T, Lee H, Matsunaga K, Kageyama S, Omori H, Noda T, et al. Atg9a controls dsDNA-driven dynamic translocation of STING and the innate immune response. Proc Natl Acad Sci USA. 2009; 106 (49): 20842-6.
Scher H I, Fizazi K, Saad F, Taplin M E, Sternberg C N, Miller K, de Wit R, Mulders P, Chi K N, Shore N D, Armstrong A J, Flaig T W, Fléchon A, Mainwaring P, Fleming M, Hainsworth J D, Hirmand M, Selby B, Seely L, de Bono J S; AFFIRM Investigators. Increased survival with enzalutamide in prostate cancer after chemotherapy. N Engl J Med. 2012; 367:1187-97.
Schlaepfer I R, Rider L, Rodrigues L U, Gijon M A, Pac C T, Romero L, Cimic A, Sirintrapun S J, Glode L M, Eckel R H, et al. Lipid catabolismvia CPT1 as a therapeutic target for prostate cancer. Mol Cancer Ther. 2014; 13 (10): 2361-71.
Schrader A J, Boegemann M, Ohlmann C H, Schnoeller T J, Krabbe L M, Hajili T, Jentzmik F, Stoeckle M, Schrader M, Herrmann E, Cronauer M V. Enzalutamide in Castration-resistant Prostate Cancer Patients Progressing After Docetaxel and Abiraterone. Eur Urol. 2013 Jul. 2. [Epub ahead of print]
Schroder K, Muruve D A, and Tschopp J. Innate immunity: cytoplasmic DNA sensing by the AIM2 inflammasome. Curr Biol. 2009; 19 (6): R262-5.
Schweizer M T, Antonarakis E S, Denmeade S R. Bipolar Androgen Therapy: A Paradoxical Approach for the Treatment of Castration-resistant Prostate Cancer. Eur Urol. 2017 Mar. 29.
Schweizer M T, Antonarakis E S, Wang H, Ajiboye A S, Spitz A, Cao H, Luo J, Haffner M C, Yegnasubramanian S, Carducci M A, Eisenberger M A, Isaacs J T, Denmeade S R. Effect of bipolar androgen therapy for asymptomatic men with castrate-resistant prostate cancer: Results from a pilot clinical study. Sci Trans Med. 2015 Jan. 7; 7 (269): 269ra2.
Sena L A, Wang H, Lim S J, Rifkind I, Ngomba N, Denmeade S R. Bipolar androgen therapy for castration-resistant prostate cancer augments response to second-generation androgen receptor signaling inhibitors. Eur J Cancer, In Press 2020.
Shah R B, Mehra R, Chinnaiyan A M, Shen R, Ghosh D, Zhou M, Macvicar G R, Varambally S, Harwood J, Bismar T A, et al. Androgen-independent prostate cancer is a heterogeneous group of diseases: lessons from a rapid autopsy program. Cancer Res 2004; 64:9209-9216.
Shang G, Zhang C, Chen Z J, Bai X C, and Zhang X. Cryo-EM structures of STING reveal its mechanism of activation by cyclic GMP-AMP. Nature. 2019; 567 (7748): 389-93.
Sheil J M, Gallimore P H, Zimmer S G, and Sopori M L. Susceptibility of Adenovirus 2-transformed rat cell lines to natural killer (NK) cells: direct correlation between NK resistance and in vivo tumorigenesis. J Immunol. 1984; 132 (3): 1578-82.
Sramkoski R M, Pretlow T G, 2nd, Giaconia J M, Pretlow T P, Schwartz S, Sy M S, Marengo S R, Rhim J S, Zhang D, and Jacobberger J W. A new human prostate carcinoma cell line, 22Rv1. In Vitro Cell Dev Biol Anim. 1999; 35 (7): 403-9.
Stockwell B R, Friedmann Angeli J P, Bayir H, Bush A I, Conrad M, Dixon S J, Fulda S, Gascon S, Hatzios S K, Kagan V E, et al. Ferroptosis: A Regulated Cell Death Nexus Linking Metabolism, Redox Biology, and Disease. Cell. 2017; 171 (2): 273-85.
Suburu J, and Chen Y Q. Lipids and prostate cancer. Prostaglandins Other Lipid Mediat. 2012; 98 (1-2): 1-10.
Sun S C. The non-canonical NF-kappaB pathway in immunity and inflammation. Nat Rev Immunol. 2017; 17 (9): 545-58.
Suzman D, Schweizer M, Luber B, Nadal R, Antonarakis E. Clinical activity of enzalutamide versus docetaxel in men with castration resistant prostate cancer progressing after abiraterone. Prostate, 2014; 74 (13): 1278-1285.
Swinnen J V, Brusselmans K, and Verhoeven G. Increased lipogenesis in cancer cells: new players, novel targets. Curr Opin Clin Nutr Metab Care. 2006; 9 (4): 358-65.
Szmulewitz R, Mohile S, Posadas E, Kunnavakkam R, Karrison T, Manchen E, Stadler W M. A Randomized Phase 1 Study of Testosterone Replacement for Patients with Low-Risk Castration-Resistant Prostate Cancer. Eur Urol. 2009 Feb. 27. [Epub ahead of print]
Taplin M E, and Balk S P. Androgen receptor: a key molecule in the progression of prostate cancer to hormone independence. J Cell Biochem. 2004; 91 (3): 483-90.
Teply B A, and Antonarakis E S. Treatment strategies for DNA repair-deficient prostate cancer. Expert Rev Clin Pharmacol. 2017; 10 (8): 889-98.
Teply B A, Kachhap S, Eisenberger M A, and Denmeade S R. Extreme Response to High-dose Testosterone in BRCA2- and ATM-mutated Prostate Cancer. Eur Urol. 2017; 71 (3): 499.
Teply B A, Wang H, Luber B, Sullivan R, Rifkind I, Bruns A, Spitz A, DeCarli M, Sinibaldi V, Pratz C F, Lu C, Silberstein J L, Luo J, Schweizer M T, Drake C G, Carducci M A, Paller C J, Antonarakis E S, Eisenberger M A, Denmeade S R. Bipolar androgen therapy in men with metastatic castration-resistant prostate cancer after progression on enzalutamide: an open-label, phase 2, multicohort study. Lancet Oncol. 2018; 19:76-86.
Thelen P, Heinrich E, Bremmer F, Trojan L, Strauss A. Testosterone boosts for treatment of castration resistant prostate cancer: An experimental implementation of intermittent androgen deprivation. Prostate 2013; 73:1699-709.
Tokunaga R, Zhang W, Naseem M, Puccini A, Berger M D, Soni S, McSkane M, Baba H, and Lenz H-J. CXCL9, CXCL10, CXCL11/CXCR3 axis for immune activation-a target for novel cancer therapy. Cancer treatment reviews. 2018; 63 (40-7.
Umekita Y, Hiipakka R A, Kokontis J M, Liao S. Human prostate tumor growth in athymic mice: Inhibition by androgens and stimulation by finasteride. Proc Natl Acad Sci USA 1996; 93:11802-07.
Vander Griend D J, Litvinov I V, and Isaacs J T. Stabilizing androgen receptor in mitosis inhibits prostate cancer proliferation. Cell Cycle. 2007; 6 (6): 647-51.
Veldscholte J, Ris-Stalpers C, Kuiper G G, Jenster G, Berrevoets C, Claassen E, et al. 1990. A mutation in the ligand binding domain of the androgen receptor of human LNCaP cells affects steroid binding characteristics and response to anti-androgens. Biochem Biophys Res Commun 1990; 173:534.
Wang W, Green M, Choi J E, Gijon M, Kennedy P D, Johnson J K, Liao P, Lang X, Kryczek I, Sell A, et al. CD8 (+) T cells regulate tumour ferroptosis during cancer immunotherapy. Nature. 2019; 569 (7755): 270-4.
Wen S, Niu Y, Lee S O, and Chang C. Androgen receptor (AR) positive vs negative roles in prostate cancer cell deaths including apoptosis, anoikis, entosis, necrosis and autophagic cell death. Cancer Treat Rev. 2014; 40 (1): 31-40.
Yin Q, Tian Y, Kabaleeswaran V, Jiang X, Tu D, Eck M J, Chen Z J, and Wu H. Cyclic di-GMP sensing via the innate immune signaling protein STING. Mol Cell. 2012; 46 (6): 735-45.
Zhang T, Dhawan, S, Healy P, George D J, Oldan J, Chin B, Armstrong A. Clinical benefit of docetaxel or enzalutamide after progression on first-line abiraterone acetate and prednisone in men with metastatic castration resistant prostate cancer (mCRPC). J Clin Onc 2014; 32: suppl abst e16031.
Zou W, Wolchok J D, and Chen L. PD-L1 (B7-H1) and PD-1 pathway blockade for cancer therapy: Mechanisms, response biomarkers, and combinations. Sci Transl Med. 2016; 8 (328): 328rv4.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating prostate cancer in a subject in need of treatment thereof, the method comprising:
(I)
(a) administering to the subject a first dose of an androgen, or a derivative thereof, at a beginning of a first treatment cycle and a second dose, and optionally a third dose, of an androgen, or a derivative thereof, at a predetermined interval during the first treatment cycle, or if the third dose is administered, at a predetermined interval during a second treatment cycle; and
(b) sequentially administering one or more antiandrogens, one or more androgen synthesis inhibitors, or a combination thereof, at a completion of the first treatment cycle and throughout the second treatment cycle, and optionally throughout a third treatment cycle; or
(II)
(a) administering to the subject a first dose of an androgen, or a derivative thereof, at a beginning of a first treatment cycle;
(b) measuring a prostate-specific antigen (PSA) level of the subject; and
(c) one of:
(i) maintaining a subject exhibiting a declining PSA level or no PSA progression on the first treatment cycle until PSA progression is observed; or
(ii) discontinuing the first treatment cycle in a subject exhibiting a PSA progression (≥25% increase in PSA from baseline) and starting a sequential treatment cycle comprising administering to the subject one or more antiandrogens, one or more androgen synthesis inhibitors, or a combination thereof.

2. The method of claim 1, wherein for the method of subparagraph (I), the second dose, or optionally the third dose, of an androgen, or a derivative thereof, is administered about 28±5 days after the beginning of the first treatment cycle or, if the third dose is administered, about 28±5 days after the beginning of the second treatment cycle.

3. The method of claim 2, further comprising starting administration of the one or more antiandrogens, one or more androgen synthesis inhibitors, or a combination thereof, at the end of a one-, two-, or three-month androgen treatment cycle.

4. The method of claim 3, comprising administering the one or more antiandrogens, one or more androgen synthesis inhibitors, or a combination thereof, each for one, two, or three 28±5 day treatment cycles.

5. The method of claim 4, further comprising discontinuing administration of the one or more antiandrogens, one or more androgen synthesis inhibitors, or a combination thereof, at a completion of the second treatment cycle, or optionally at a completion of the third treatment cycle, and restarting the first treatment cycle comprising administering to the subject a first dose of an androgen, or a derivative thereof, at a beginning of a first treatment cycle and a second dose, and optionally a third dose, of an androgen, or a derivative thereof, at a predetermined interval during the first treatment cycle, or if the third dose is administered, at a predetermined interval during a second treatment cycle.

6. The method of claim 5, comprising alternating the first treatment cycle and the second treatment cycle, and optionally the third treatment cycle, until a clinical and/or radiographic progression is observed.

7. (canceled)

8. The method of claim 1, for the method of subparagraph (II), further comprising continuing administration of the one or more antiandrogens, one or more androgen synthesis inhibitors, or a combination thereof, until PSA progression is observed.

9. The method of claim 8, further comprising discontinuing administration of the one or more antiandrogens, one or more androgen synthesis inhibitors, or a combination thereof, once the subject exhibits PSA progression (≥25% increase in PSA from baseline) and restarting the first treatment cycle.

10. The method of claim 9, further comprising alternating between the first treatment cycle and the second treatment cycle, and optionally the third treatment cycle, with onset of PSA progression until clinical and/or radiographic progression is observed.

11. The method of claim 1, wherein the first dose of an androgen, or a derivative thereof, and the second dose of an androgen, or a derivative thereof, and optionally the third dose of an androgen, or a derivative thereof, are each sufficient to achieve a supraphysiological serum concentration of testosterone in the subject.

12. The method of claim 11, wherein the supraphysiological serum concentration of testosterone in the subject is between about 3 to about 10 times a normal serum concentration of testosterone.

13. The method of claim 12, wherein the serum concentration of testosterone is greater than about 1,500 ng/dL.

14. The method of claim 1, wherein the androgen, or derivative thereof, is testosterone cypionate or testosterone enanthate at a dose of about 400 to about 500 mg.

15. The method of claim 1, wherein one or more androgens having a different biological potency are administered to the subject, wherein the first dose of an androgen, or a derivative thereof, and the second dose of an androgen, or a derivative thereof, and optionally a third dose of an androgen, or a derivative thereof, are given at a dose range that achieves a same relative supraphysiologic potency as that achieved with testosterone cypionate or testosterone enanthate a dose of about 400 mg to about 500 mg.

16. The method of claim 1, wherein the method comprises administering the androgen, or a derivative thereof, orally, transdermally or by intramuscular injection.

17. The method of claim 1, wherein the androgen, or a derivative thereof, comprises an ester of testosterone or an ester of dihydrotestosterone.

18. The method of claim 17, wherein the ester of testosterone of the ester of dihydrotestosterone is selected from a cypionate, enanthate, propionate, butyrate, and undecanoate ester of testosterone or dihydrotestosterone.

19. The method of claim 18, wherein the androgen, or derivative thereof, is testosterone cypionate or testosterone enanthate.

20. The method of claim 1, wherein the one or more antiandrogens is selected from the group consisting of bicalutamide, flutamide, nilutamide, apalutamide, darolutamide, enzalutamide, cyproterone acetate, proxalutamide, cimetidine, and topilutamide, and combinations thereof.

21. (canceled)

22. (canceled)

23. The method of claim 1, wherein the one or more androgen synthesis inhibitors is selected from the group consisting of a CYP17A1 inhibitor, a CYP11A1 (P450scc) inhibitor, a 5α-Reductase inhibitor, and combinations thereof.

24. The method of claim 23, wherein the one or more androgen synthesis inhibitors is selected from the group consisting of abiraterone acetate, ketoconazole, seviteronel, aminoglutethimide, alfatradiol, dutasteride, epristeride, finasteride, and combinations thereof.

25. (canceled)

26. The method of claim 1, wherein the one or more antiandrogens, one or more androgen synthesis inhibitors, or a combination thereof, are administered at a dosage having a range selected from the group between about 100 to about 200 mg/day, between about 110 to about 190 mg/day, between about 120 to about 180 mg/day, between about 130 to about 170 mg/day, and about 160 mg per day.

27. The method of claim 1, further comprising concurrently administering an androgen deprivation therapy (ADT) to the subject.

28. The method of claim 27, wherein the ADT comprises surgical castration or administering a luteinizing hormone-releasing hormone (LHRH) agonist or a LHRH antagonist to the subject.

29. The method of claim 28, wherein the LHRH agonist is selected from the group consisting of leuprolide, goserelin, triptorelin, and histrelin.

30. The method of claim 29, wherein the LHRH antagonist is selected from the group consisting of degarelix and relugolix.

31. The method of claim 1, further comprising administering immune checkpoint blockade therapy to the subject if the subject exhibits clinical and/or radiographic progression.

32. The method of claim 31, wherein the immune checkpoint blockade therapy comprises administering an anti-PD1/PDL1 antibody or an anti-CTLA4 antibody.

33. The method of claim 32, wherein the anti-PD1/PDL1 antibody is selected from the group consisting of pembrolizumab, nivolumab, and atezolizumab.

34. The method of claim 32, wherein the anti-CTLA4 antibody comprises ipilimumab.

35. The method of claim 1, wherein the subject has progressive prostate cancer after treatment with abiraterone in combination with androgen deprivation therapy (ADT) as an initial therapy or as a second-line therapy after development of resistance to primary ADT.

36. The method of claim 1, wherein the prostate cancer comprises castration resistant metastatic prostate cancer.

37. The method of claim 1, wherein the subject is asymptomatic.

38. The method of claim 1, wherein the subject is symptomatic.
